(19) United States
(12) Patent Application Publication
(10) Pub. No.: US 2005/0256154 A1
(43) Pub. Date: Nov. 17, 2005

(54) 4-Amino-thieno[3,2-c]pyridine-7-carboxylic acid amides

(76) Inventors: Kin-Chun Luk, North Caldwell, NJ (US); Lee Apostle McDermott, Parlin, NJ (US); Pamela Loreen Rossman, Nutley, NJ (US); Peter Michael Wovkulich, Nutley, NJ (US); Zhuming Zhang, Hillsborough, NJ (US)

Correspondence Address: HOFFMANN-LA ROCHE INC.;PATENT LAW DEPARTMENT, 340 KINGSLAND STREET, NUTLEY, NJ, 07110, US

(21) Appl. No.: 11/110,614

(22) Filed: Apr. 20, 2005

Related U.S. Application Data
(60) Provisional application No. 60/568,047, filed on May 4, 2004.
(60) Provisional application No. 60/618,795, filed on Oct. 14, 2004.

(51) Int. Cl. A61K 31/4743; C07D 498/02

(52) U.S. Cl. 514/301; 546/114

(57) ABSTRACT

Disclosed are novel 4-amino-thieno[3,2-c]pyridine-7-carboxylic acid amides, and their pharmaceutically acceptable salts and esters, that are selective inhibitors of KDR and/or FGFR kinases. These compounds and their pharmaceutically acceptable salts are anti-proliferative agents useful in the treatment or control of solid tumors, in particular solid cancerous tumors of the breast, colon, lung and prostate. Also disclosed are pharmaceutical compositions containing these compounds and methods of treating cancer using these compounds.

### PRIORITY TO RELATED APPLICATIONS

This application claims the benefit of Provisional Application Ser. No. 60/568,047, filed May 4, 2004 and Ser. No. 60/618,795, filed Oct. 14, 2004.

### FIELD OF THE INVENTION

The present invention is directed to novel 4-amino-thieno[3,2-c]pyridine-7-carboxylic acid amides and their pharmaceutically acceptable salts and esters. These compounds inhibit KDR (kinase insert domain-containing receptor) kinase and/or FGFR (fibroblast growth factor receptor) kinase. These compounds and their pharmaceutically acceptable salts and esters have antiproliferative activity and are useful in the treatment or control of cancer, in particular solid tumors. In addition these compounds have advantageous bioavailability profiles. This invention is also directed to pharmaceutical compositions containing such compounds and to methods of treating or controlling cancer, most particularly the treatment or control of breast, lung, colon and prostate tumors.

### BACKGROUND OF THE INVENTION

Protein kinases are a class of proteins (enzymes) that regulate a variety of cellular functions. This is accomplished by the phosphorylation of specific amino acids on protein substrates resulting in conformational alteration of the substrate protein. The conformational change modulates the activity of the substrate or its ability to interact with other binding partners. The enzyme activity of the protein kinase refers to the rate at which the kinase adds phosphate groups to a substrate. It can be measured, for example, by determining the amount of a substrate that is converted to a product as a function of time. Phosphorylation of a substrate occurs at the active-site of a protein kinase.

Tyrosine kinases are a subset of protein kinases that catalyze the transfer of the terminal phosphate of adenosine triphosphate (ATP) to tyrosine residues on protein substrates. These kinases play an important part in the propagation of growth factor signal transduction that leads to cellular proliferation, differentiation and migration.

For example, basic fibroblast growth factor (FGF) and vascular endothelial growth factor (VEGF) have been recognized as important mediators of tumor promoted angiogenesis. VEGF activates endothelial cells by signaling through two high affinity receptors, one of which is the kinase insert domain-containing receptor (KDR). See Hennequin L. F. et. al., J. Med. Chem. 2002, 45(6), pp 1300. FGF activates endothelial cells by signaling through the FGF receptor (FGFR). Solid tumors depend upon the formation of new blood vessels (angiogenesis) to grow. Accordingly, inhibitors of the receptors FGFR and/or KDR that interfere with the growth signal transduction, and thus slow down or prevent angiogenesis, are useful agents in the prevention and treatment of solid tumors. See Klohs W. E. et. al., Current Opinion in Biotechnology 1999, 10, p. 544.

There is a need for easily synthesized, small-molecule compounds effective in inhibiting the catalytic activity of protein kinases, in particular FGFR and KDR kinases, for treating one or more types of solid tumors. It is particularly desirable to provide small molecule inhibitors that are selective for FGFR and/or KDR. This is desirable because of the potential concomitant toxicity and other undesirable complications that may follow from inhibiting multiple targets. It is preferable that such small molecule inhibitors also possess advantageous bioavailability profiles. It is thus an object of this invention to provide such compounds and pharmaceutical compositions containing these compounds.

### SUMMARY OF THE INVENTION

In one embodiment, the present invention relates to novel 4-amino-thieno[3,2-c]pyridine-7-carboxylic acid amides capable of selectively inhibiting the activity of KDR and/or FGFR. These compounds are useful in the treatment or control of cancer, in particular the treatment or control of solid tumors. In particular this invention relates to compounds of formula 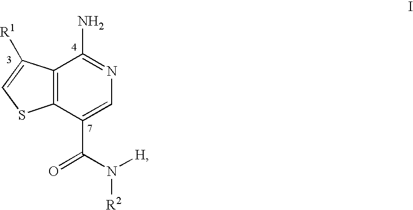
or the pharmaceutically acceptable salts and esters thereof, wherein R¹ and R² are as hereinafter defined.

The present invention also relates to pharmaceutical compositions comprising a therapeutically effective amount of one or more compounds of formula I, or a pharmaceutically acceptable salt or ester thereof, and a pharmaceutically acceptable carrier or excipient.

The present invention further relates to a method for treating or controlling solid tumors, in particular treatment or control of breast, lung, colon and prostate tumors, most particularly breast or colon tumors, by administering to a human patient in need of such therapy an effective amount of a compound of formula I and/or a pharmaceutically acceptable salt thereof.

The present invention is further directed to novel intermediate compounds useful in the preparation of compounds of formula I.

### DETAILED DESCRIPTION OF THE INVENTION

Definitions

As used herein, the following terms shall have the following definitions.

“Alkyl” denotes a straight-chain or branched saturated aliphatic hydrocarbon having 1 to 10, preferably 1 to 6, and more preferably 1 to 4 carbon atoms. Alkyl groups having 1 to 6 carbon atoms are also referred to herein as “lower alkyl.” Typical lower alkyl groups include methyl, ethyl, propyl, isopropyl, butyl, t-butyl, 2-butyl, pentyl and hexyl. As used herein the sample designation C₁₋₄ alkyl means alkyl having from 1 to 4 carbon atoms.

“Aryl” means an aromatic carbocyclic radical, for example a 6-10 membered aromatic or partially aromatic ring system. Preferred aryl groups include, but are not limited to, phenyl, naphthyl, tolyl and xylyl.

“Cycloalkyl” means a non-aromatic, partially or completely saturated cyclic aliphatic hydrocarbon group containing 3 to 8 atoms. Examples of cycloalkyl groups include cyclopropyl, cyclopentyl and cyclohexyl.

“Effective amount” or “Therapeutically effective amount” means an amount of at least one compound for formula I, or a pharmaceutically acceptable salt thereof, that significantly inhibits tumor growth.

“Halogen” means fluorine, chlorine, bromine or iodine, preferably bromine, chlorine or fluorine.

“Hetero atom” means an atom selected from N, O and S, preferably N. [If the hetero atom is N, it can be present as —NH— or —N-lower alkyl-. If the hetero atom is S, it can be present as S, SO or SO₂.

“Heteroaryl” means an aromatic heterocyclic ring system containing up to two rings. Preferred heteroaryl groups include, but are not limited to, thienyl, furyl, indolyl, pyrrolyl, pyridinyl, pyrazinyl, oxazolyl, thiaxolyl, quinolinyl, pyrimidinyl, imidazolyl and tetrazolyl.

“Heterocycle” or “heterocyclyl” means a 3- to 10-membered saturated or partially unsaturated non-aromatic monovalent cyclic radical having from one to 3 hetero atoms selected from nitrogen, oxygen or sulfur or a combination thereof. Examples of preferred heterocycles are piperidine, piperazine, pyrrolidine, and morpholine.

“IC₅₀” refers to the concentration of a particular compound according to the invention required to inhibit 50% of a specific measured activity. IC₅₀ can be measured, inter alia, as is described in Example 26, infra.

“Pharmaceutically acceptable ester” refers to a conventionally esterified compound of formula I having a carboxyl group, which esters retain the biological effectiveness and properties of the compounds of formula I and are cleaved in vivo (in the organism) to the corresponding active carboxylic acid. Examples of ester groups which are cleaved (in this case hydrolyzed) in vivo to the corresponding carboxylic acids (R⁴⁰C(═O)OH) are lower alkyl esters which may be substituted with NR⁴¹R⁴² where R⁴¹ and R⁴² are lower alkyl, or where NR⁴¹R⁴² taken together form a monocyclic aliphatic heterocycle, such as pyrrolidine, piperidine, morpholine, N-methylpiperazine, etc.; acyloxyalkyl esters of the formula R⁴⁰C(═O)OCHR⁴³OC(═O)R⁴⁴ where R⁴³ is hydrogen or methyl, and R⁴⁴ is lower alkyl or cycloalkyl; carbonate esters of the formula R⁴⁰C(═O)OCHR⁴³OC(═O)OR⁴⁵ where R⁴³ is hydrogen or methyl, and R⁴⁵ is lower alkyl or cycloalkyl; or aminocarbonylmethyl esters of the formula R⁴⁰C(═O)OCH₂C(═O)NR⁴¹R⁴² where R⁴¹ and R⁴² are hydrogen or lower alkyl, or where NR⁴¹R⁴² taken together form a monocyclic aliphatic heterocycle, such as pyrrolidine, piperidine, morpholine, N-methylpiperazine, etc.

Examples of lower alkyl esters are the methyl, ethyl, and n-propyl esters, and the like. Examples of lower alkyl esters substituted with NR⁴¹R⁴² are the diethylaminoethyl, 2-(4-morpholinyl)ethyl, 2-(4-methylpiperazin-1-yl)ethyl esters, and the like. Examples of acyloxyalkyl esters are the pivaloxymethyl, 1-acetoxyethyl, and acetoxymethyl esters. Examples of carbonate esters are the 1-(ethoxycarbonyloxy)ethyl and 1-(cyclohexyloxycarbonyloxy)ethyl esters. Examples of aminocarbonylmethyl esters are the N,N-dimethylcarbamoylmethyl and carbamoylmethyl esters.

Further information concerning examples of and the use of esters for the delivery of pharmaceutical compounds is available in Design of Prodrugs. Bundgaard H ed. (Elsevier, 1985). See also, H. Ansel et. al., Pharmaceutical Dosage Forms and Drug Delivery Systems (6th Ed. 1995) at pp. 108-109; Krogsgaard-Larsen, et. al., Textbook of Drug Design and Development (2d Ed. 1996) at pp. 152-191.

“Pharmaceutically acceptable salt” refers to conventional acid-addition salts or base-addition salts that retain the biological effectiveness and properties of the compounds of formula I and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Sample base-addition salts include those derived from ammonium, potassium, sodium and, quaternary ammonium hydroxides, such as for example, tetramethylammonium hydroxide. The chemical modification of a pharmaceutical compound (i.e. drug) into a salt is a technique well known to pharmaceutical chemists to obtain improved physical and chemical stability, hygroscopicity, flowability and solubility of compounds. See, e.g., H. Ansel et. al., Pharmaceutical Dosage Forms and Drug Delivery Systems (6th Ed. 1995) at pp. 196 and 1456-1457.

“Pharmaceutically acceptable,” such as pharmaceutically acceptable carrier, excipient, etc., means pharmacologically acceptable and substantially non-toxic to the subject to which the particular compound is administered.

“Substituted,” as in substituted alkyl, means that the substitution can occur at one or more positions and, unless otherwise indicated, that the substituents at each substitution site are independently selected from the specified options.

In one embodiment, the invention relates to compounds of formula 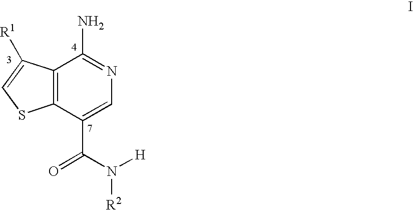
wherein
R¹ is selected from
   lower alkyl, and
   lower alkyl substituted with OR³, NR³R⁴, S(O)NR³, cycloalkyl, substituted cycloalkyl, heterocycle, substituted heterocycle, heteroaryl, or substituted heteroaryl;
R² is selected from
   H,
   lower alkyl, and
   lower alkyl substituted with OR⁵, OC(O)R⁵, NR⁵R⁶, S(O)ₙR⁵, aryl, substituted aryl, cycloalkyl, substituted cycloalkyl, heterocycle, substituted heterocycle, heteroaryl, or substituted heteroaryl;
R³ and R⁴ are independently selected from
   H,
   lower alkyl,
   lower alkyl substituted with aryl, aryl fused to a heterocycle or a substituted heterocycle, substituted aryl, heteroaryl, substituted heteroaryl, cycloalkyl, substituted cycloalkyl, heterocycle, or substituted heterocycle,
   aryl,
   aryl fused to a heterocycle or a substituted heterocycle,
   substituted aryl,
   heteroaryl,
   heteroaryl fused to a heterocycle or a substituted heterocycle,
   substituted heteroaryl,
   heterocycle,
   heterocycle fused to an aryl,
   cycloalkyl, and
   substituted cycloalkyl,
   or, alternately, the group NR³R⁴ independently can form a ring having a total of 3 to 7 atoms, said ring atoms comprising in addition to the nitrogen to which R³ and R⁴ are bonded, carbon ring atoms, said carbon ring atoms optionally being replaced by one or more additional heteroatoms, and said ring atoms optionally being substituted by the group consisting of one or more lower alkyl, ═O, OR⁷, COR⁷, CO₂R⁷, CONR⁷R⁸, SOₙR⁷, and SO₂NR⁷R⁸;
R⁵ and R⁶ are independently selected from
   H,
   lower alkyl, and
   lower alkyl substituted with OR⁷, NR⁷R⁸, aryl, substituted aryl, heteroaryl, substituted heteroaryl, heterocycle, cycloalkyl, substituted cycloalkyl,
   or, alternately, the group NR⁵R⁶ independently can form a ring having a total of 3 to 7 atoms, said ring atoms comprising in addition to the nitrogen to which R⁵ and R⁶ are bonded, carbon ring atoms, said carbon ring atoms optionally being replaced by one or more additional heteroatoms, and said ring atoms optionally being substituted by the group consisting of one or more lower alkyl, ═O, OR⁷, NR⁷R⁸, COR⁷, CO₂R⁷, CONR⁷R⁸, SOₙR⁷, and SO₂NR⁷R⁸;
R⁷ and R⁸ are independently selected from
   H, lower alkyl, aryl or heteroaryl,
or, alternatively, the group NR⁷R⁸ independently can form a ring having a total of 3 to 7 atoms, said ring atoms comprising in addition to the nitrogen to which R⁷ and R⁸ are bonded, carbon ring atoms, said carbon ring atoms optionally being replaced by one or more additional heteroatoms, and said ring atoms optionally being substituted by the group consisting of one or more lower alkyl, ═O, or OR⁹;
R⁹ is H or lower alkyl; and
n is 0, 1 or 2;
wherein,
substituted aryl and substituted heteroaryl are aryl and heteroaryl that are substituted with one or more groups independently selected from lower alkyl, OR⁷, NR⁷R⁸, COR⁷, CO₂R⁷, CONR⁷R⁸, SO₂NR⁷R⁸, SOₙR⁷, CN, NO₂, and halogen; and
substituted cycloalkyl and substituted heterocycle are cycloalkyl and heterocycle that are substituted with one or more groups independently selected from lower alkyl, ═O, OR⁷, NR⁷R⁸, COR⁷, CO₂R⁷, CONR⁷R⁸, SO₂NR⁷R⁸, SOₙR⁷, and CN;
or a pharmaceutically acceptable salt or ester thereof.

Compounds disclosed herein and covered by formula I above may exhibit tautomerism or structural isomerism. It is intended that the invention encompasses any tautomeric or structural isomeric form of these compounds, or mixtures of such forms (e.g. racemic mixtures), and is not limited to any one tautomeric or structural isomeric form depicted in formula I above.

One skilled in the art would understand that the groups NR³R⁴, NR⁵R⁶ and NR⁷R⁸ as defined above may include one or more ring heteroatoms in addition to the above-mentioned N. The total number of additional ring heteroatoms, that is in addition to the above-mentioned N, depends on the particular ring system involved. Preferably, there are no more than 1 or 2 additional ring heteroatoms.

In one embodiment, the invention relates to a compound of formula I wherein R¹ is lower alkyl substituted with OR³. Preferred R³ groups include aryl, aryl substituted with halogen, and aryl fused to a heterocycle. Preferred halogen groups include Br, Cl and F.

In another embodiment, the invention relates to a compound of formula I wherein R¹ is lower alkyl substituted with OR³. Preferred R³ groups include heteroaryl and heteroaryl substituted with OR⁷.

In another embodiment, the invention relates to a compound of formula I wherein R¹ is lower alkyl substituted with NR³R⁴. Preferably, the group NR³R⁴ forms a ring having a total of 3 to 7 ring atoms comprising in addition to the nitrogen to which R³ and R⁴ are bonded, carbon ring atoms, said carbon ring atoms optionally being replaced by one or more additional heteroatoms, and said ring atoms optionally being substituted by the group consisting of one or more lower alkyl, ═O, OR⁷, COR⁷, CO₂R⁷, CONR⁷R⁸, SOₙR⁷, and SO₂NR⁷R⁸, preferably OR⁷. Most preferably said ring atoms are unsubstituted or substituted by lower alkyl and ═O.

In another embodiment, the invention relates to a compound of formula I wherein R¹ is lower alkyl substituted with S(O)NR³, wherein R³ is lower alkyl.

In another embodiment, the invention relates to a compound of formula I wherein R¹ is lower alkyl substituted with cycloalkyl.

In another embodiment, the invention relates to a compound of formula I wherein R¹ is lower alkyl substituted with substituted cycloalkyl.

In another embodiment, the invention relates to a compound of formula I wherein R¹ is lower alkyl substituted with heterocycle.

In another embodiment, the invention relates to a compound of formula I wherein R¹ is lower alkyl substituted with substituted heterocycle.

In another embodiment, the invention relates to a compound of formula I wherein R¹ is lower alkyl substituted with heteroaryl.

In another embodiment, the invention relates to a compound of formula I wherein R¹ is lower alkyl substituted with substituted heteroaryl.

In another embodiment, the invention relates to a compound of formula I wherein R¹ is lower alkyl.

In another embodiment, the invention relates to a compound of formula I wherein R² is lower alkyl substituted with OR⁵ wherein R⁵ is lower alkyl substituted with NR⁷R⁸.

In another embodiment, the invention relates to a compound of formula I wherein R² is lower alkyl substituted with NR⁵R⁶. Preferably, the group NR⁵R⁶ forms a ring having a total of 3 to 7 ring atoms, said ring atoms comprising in addition to the nitrogen to which R⁵ and R⁶ are bonded, carbon ring atoms, said carbon ring atoms optionally being replaced by one or more additional heteroatoms, and said ring atoms optionally being substituted by the group consisting of one or more lower alkyl, ═O, OR⁷, NR⁷R⁸, COR⁷, CO₂R⁷, CONR⁷R⁸, SOₙR⁷, and SO₂NR⁷R⁸. Most preferably said ring atoms are unsubstituted or substituted by lower alkyl, ═O, and OR⁷.

In another embodiment, the invention relates to a compound of formula I wherein R² is lower alkyl.

In another embodiment, the invention relates to a compound of formula I wherein R² is lower alkyl substituted with one or more OH groups or one NR⁵R⁶ group.

In another embodiment, the invention relates to a compound of the formula I wherein R² is lower alkyl substituted by OR⁵.

In another embodiment, the invention relates to a compound of formula I wherein R² is lower alkyl substituted with OC(O)R⁵.

In another embodiment, the invention relates to a compound of formula I wherein R² is lower alkyl substituted with S(O)NR⁵ wherein R⁵ is lower alkyl and n is 1 or 2.

In another embodiment, the invention relates to a compound of formula I wherein R² is lower alkyl substituted with aryl.

In another embodiment, the invention relates to a compound of formula I wherein R² is lower alkyl substituted with substituted aryl.

In another embodiment, the invention relates to a compound of formula I wherein R² is lower alkyl substituted with cycloalkyl.

In another embodiment, the invention relates to a compound of formula I wherein R² is lower alkyl substituted with substituted cycloalkyl.

In another embodiment, the invention relates to a compound of formula I wherein R² is lower alkyl substituted with heterocycle.

In another embodiment, the invention relates to a compound of formula I wherein R² is lower alkyl substituted with substituted heterocycle.

In another embodiment, the invention relates to a compound of formula I wherein R² is lower alkyl substituted with heteroaryl.

In another embodiment, the invention relates to a compound of formula I wherein R² is lower alkyl substituted with substituted heteroaryl.

In another embodiment, the invention relates to a compound of formula I wherein R² is H.

In another embodiment, the invention relates to a compound of formula I wherein R³ is H.

In another embodiment, the invention relates to a compound of formula I wherein R³ is lower alkyl.

In another embodiment, the invention relates to a compound of formula I wherein R³ is lower alkyl substituted with aryl, aryl fused to a heterocycle or a substituted heterocycle, substituted aryl, heteroaryl, substituted heteroaryl, cycloalkyl, substituted cycloalkyl, heterocycle, or substituted heterocycle.

In another embodiment, the invention relates to a compound of formula I wherein R³ is aryl.

In another embodiment, the invention relates to a compound of formula I wherein R³ is aryl fused to a heterocycle or a substituted heterocycle.

In another embodiment, the invention relates to a compound of formula I wherein R³ is substituted aryl.

In another embodiment, the invention relates to a compound of formula I wherein R³ is heteroaryl.

In another embodiment, the invention relates to a compound of formula I wherein R³ is heteroaryl fused to a heterocycle or a substituted heterocycle.

In another embodiment, the invention relates to a compound of formula I wherein R³ is substituted heteroaryl.

In another embodiment, the invention relates to a compound of formula I wherein R³ is heterocycle.

In another embodiment, the invention relates to a compound of formula I wherein R³ is heterocycle fused to an aryl.

In another embodiment, the invention relates to a compound of formula I wherein R³ is cycloalkyl.

In another embodiment, the invention relates to a compound of formula I wherein R³ is substituted cycloalkyl.

In another embodiment, the invention relates to a compound of formula I wherein R³ is lower alkyl, heterocycle fused to an aryl, aryl, substituted aryl, or aryl fused to a heterocycle.

In another embodiment, the invention relates to a compound of formula I wherein R⁴ is H.

In another embodiment, the invention relates to a compound of formula I wherein R⁴ is lower alkyl.

In another embodiment, the invention relates to a compound of formula I wherein R⁴ is lower alkyl substituted with aryl, aryl fused to a heterocycle or a substituted heterocycle, substituted aryl, heteroaryl, substituted heteroaryl, cycloalkyl, substituted cycloalkyl, heterocycle, or substituted heterocycle.

In another embodiment, the invention relates to a compound of formula I wherein R⁴ is aryl.

In another embodiment, the invention relates to a compound of formula I wherein R⁴ is aryl fused to a heterocycle or a substituted heterocycle.

In another embodiment, the invention relates to a compound of formula I wherein R⁴ is substituted aryl.

In another embodiment, the invention relates to a compound of formula I wherein R⁴ is heteroaryl.

In another embodiment, the invention relates to a compound of formula I wherein R⁴ is heteroaryl fused to a heterocycle or a substituted heterocycle.

In another embodiment, the invention relates to a compound of formula I wherein R⁴ is substituted heteroaryl.

In another embodiment, the invention relates to a compound of formula I wherein R⁴ is heterocycle.

In another embodiment, the invention relates to a compound of formula I wherein R⁴ is heterocycle fused to an aryl.

In another embodiment, the invention relates to a compound of formula I wherein R⁴ is cycloalkyl.

In another embodiment, the invention relates to a compound of formula I wherein R⁴ is substituted cycloalkyl.

In another embodiment, the invention relates to a compound of formula I wherein the group NR³R⁴ forms a ring having a total of 3 to 7 atoms, said ring atoms comprising in addition to the nitrogen to which R³ and R⁴ are bonded, carbon ring atoms, said carbon ring atoms optionally being replaced by one or more additional heteroatoms, and said ring atoms optionally being substituted by the group consisting of one or more lower alkyl, ═O, OR⁷, COR⁷, CO₂R⁷, CONR⁷R⁸, SOₙR⁷, and SO₂NR⁷R⁸. Most preferably said ring atoms are unsubstituted or substituted by lower alkyl, ═O and OR⁷.

In another embodiment, the invention relates to a compound of formula I wherein R⁵ is H.

In another embodiment, the invention relates to a compound of formula I wherein R⁵ is lower alkyl.

In another embodiment, the invention relates to a compound of formula I wherein R⁵ is lower alkyl substituted with NR⁷R⁸, aryl, substituted aryl, heteroaryl, substituted heteroaryl, heterocycle, cycloalkyl, substituted cycloalkyl. Most preferably R⁵ is lower alkyl substituted with NR⁷R⁸.

In another embodiment, the invention relates to a compound of formula I wherein R⁵ is lower alkyl substituted by one or more OR⁷.

In another embodiment, the invention relates to a compound of formula I wherein R⁶ is H.

In another embodiment, the invention relates to a compound of formula I wherein R⁶ is lower alkyl.

In another embodiment, the invention relates to a compound of formula I wherein R⁶ is lower alkyl substituted with NR⁷R⁸, aryl, substituted aryl, heteroaryl, substituted heteroaryl, heterocycle, cycloalkyl, substituted cycloalkyl. Most preferably R⁶ is lower alkyl substituted with NR⁷R⁸.

In another embodiment, the invention relates to a compound of formula I wherein the group NR⁵R⁶ forms a ring having a total of 3 to 7 atoms, said ring atoms comprising in addition to the nitrogen to which R⁵ and R⁶ are bonded, carbon ring atoms, said carbon ring atoms optionally being replaced by one or more additional heteroatoms, and said ring atoms optionally being substituted by the group consisting of one or more lower alkyl, ═O, OR⁷, NR⁷R⁸, COR⁷, CO₂R⁷, CONR⁷R⁸, SOₙR⁷, and SO₂NR⁷R⁸. Most preferably said ring atoms are unsubstituted or substituted with lower alkyl, ═O and OR⁷.

In another embodiment, the invention relates to a compound of formula I wherein R⁷ is H.

In another embodiment, the invention relates to a compound of formula I wherein R⁷ is lower alkyl.

In another embodiment, the invention relates to a compound of formula I wherein R⁸ is H.

In another embodiment, the invention relates to a compound of formula I wherein R⁸ is lower alkyl.

In another embodiment, the invention relates to a compound of formula I wherein the group NR⁷R⁸ forms a ring having a total of 3 to 7 atoms, said ring atoms comprising in addition to the nitrogen to which R⁷ and R⁸ are bonded, carbon ring atoms, said carbon ring atoms optionally being replaced by one or more additional heteroatoms, and said ring atoms optionally being substituted by the group consisting of one or more lower alkyl, ═O, or OR⁹.

In another embodiment, the invention relates to a compound of formula I wherein R⁸ is H.

In another embodiment, the invention relates to a compound of formula I wherein R⁸ is lower alkyl.

In another embodiment, the invention relates to a compound of formula I wherein R⁹ is H.

In another embodiment, the invention relates to a compound of formula I wherein R⁹ is lower alkyl.

In another embodiment, the invention relates to a compound of formula I 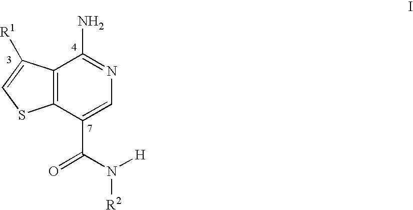
wherein
R¹ is lower alkyl substituted with OR³;
R² is H or lower alkyl substituted with one or more OR⁵ groups or one NR⁵R⁶ group;
R³ is aryl substituted with halogen or OR⁷, or is aryl fused to a heterocycle;
R⁵ and R⁶ are independently H, lower alkyl or lower alkyl substituted by one or more OR⁷, or alternatively, the group NR⁵R⁶ independently can form a ring having a total of from 3 to 6 atoms, said ring atoms comprising in addition to the nitrogen to which R⁵ and R⁶ are bonded, carbon ring atoms, said carbon ring atoms optionally being replaced by one additional heteroatoms selected from N or O, and said ring atoms optionally being substituted by OR⁷; and
R⁷ is H or lower alkyl;
or a pharmaceutically acceptable salt or ester thereof.

The following compounds are preferred embodiments according to the present invention:
4-Amino-3-(4-bromo-2,6-difluoro-phenoxymethyl)-thieno[3,2-c]pyridine-7-carboxylic acid (2-hydroxy-ethyl)-amide (Example 1),
4-Amino-3-(2-chloro-4-methoxy-phenoxymethyl)-thieno[3,2-c]pyridine-7-carboxylic acid (2-hydroxy-ethyl)-amide (Example 2),
4-Amino-3-(benzo[1,3]dioxol-5-yloxymethyl)-thieno[3,2-c]pyridine-7-carboxylic acid (2-hydroxy-ethyl)-amide (Example 3),
4-Amino-3-(4-bromo-phenoxymethyl)-thieno[3,2-c]pyridine-7-carboxylic acid (2-hydroxy-ethyl)-amide (Examples 4a and 4b),
4-Amino-3-phenoxymethyl-thieno[3,2-c]pyridine-7-carboxylic acid (2-hydroxy-ethyl)-amide (Example 5),
rac-4-Amino-3-(4-bromo-phenoxymethyl)-thieno[3,2-c]pyridine-7-carboxylic acid (2-hydroxy-1-methyl-ethyl)-amide (Example 6),
rac-4-Amino-3-(4-bromo-phenoxymethyl)-thieno[3,2-c]pyridine-7-carboxylic acid (2-hydroxy-propyl)-amide (Example 7),
rac-4-Amino-3-(4-bromo-phenoxymethyl)-thieno[3,2-c]pyridine-7-carboxylic acid (2,3-dihydroxy-propyl)-amide (Example 8),
4-Amino-3-(4-bromo-phenoxymethyl)-thieno[3,2-c]pyridine-7-carboxylic acid (2-hydroxy-1,1-dimethyl-ethyl)-amide (Example 9),
4-Amino-3-(4-bromo-phenoxymethyl)-thieno[3,2-c]pyridine-7-carboxylic acid (2-hydroxy-1-hydroxymethyl-ethyl)-amide (Example 10),
4-Amino-3-(4-bromo-phenoxymethyl)-thieno[3,2-c]pyridine-7-carboxylic acid (2-diethylamino-ethyl)-amide (Example 11),
4-Amino-3-(4-bromo-phenoxymethyl)-thieno[3,2-c]pyridine-7-carboxylic acid (4-pyrrolidin-1-yl-butyl)-amide (Example 12a),
4-Amino-3-(4-bromo-phenoxymethyl)-thieno[3,2-c]pyridine-7-carboxylic acid (4-pyrrolidin-1-yl-butyl)-amide hydrochloride salt (Example 12b),
4-Amino-3-(4-bromo-phenoxymethyl)-thieno[3,2-c]pyridine-7-carboxylic acid (4-pyrrolidin-1-yl-butyl)-amide methanesulfonic acid salt (Example 12c),
4-Amino-3-(4-bromo-phenoxymethyl)-thieno[3,2-c]pyridine-7-carboxylic acid (2-morpholin-4-yl-ethyl)-amide (Example 13a)
4-Amino-3-(4-bromo-phenoxymethyl)-thieno[3,2-c]pyridine-7-carboxylic acid (2-morpholin-4-yl-ethyl)-amide hydrochloride salt (Example 13b),
4-Amino-3-(4-bromo-phenoxymethyl)-thieno[3,2-c]pyridine-7-carboxylic acid (3-dimethylamino-2,2-dimethyl-propyl)-amide (Example 14),
rac-4-Amino-3-(4-bromo-phenoxymethyl)-thieno[3,2-c]pyridine-7-carboxylic acid (5-diethylamino-1-methyl-pentyl)-amide (Example 15),
4-Amino-3-(4-bromo-phenoxymethyl)-thieno[3,2-c]pyridine-7-carboxylic acid amide (Example 16),
4-Amino-3-(4-bromo-phenoxymethyl)-thieno[3,2-c]pyridine-7-carboxylic acid [2-(2-pyrrolidin-1-yl-ethoxy)-ethyl]-amide (Example 17),
4-Amino-3-(4-bromo-phenoxymethyl)-thieno[3,2-c]pyridine-7-carboxylic acid [4-(3-methoxy-pyrrolidin-1-yl)-butyl]-amide (Example 18),
4-Amino-3-(4-bromo-phenoxymethyl)-thieno[3,2-c]pyridine-7-carboxylic acid (4-piperidin-1-yl-butyl)-amide (Example 19),
4-Amino-3-(4-bromo-phenoxymethyl)-thieno[3,2-c]pyridine-7-carboxylic acid [2-(2-piperidin-1-yl-ethoxy)-ethyl]-amide (Example 20),
4-Amino-3-(4-bromo-phenoxymethyl)-thieno[3,2-c]pyridine-7-carboxylic acid [4-(3-methoxy-piperidin-1-yl)-butyl]-amide (Example 21),
4-Amino-3-(4-bromo-phenoxymethyl)-thieno[3,2-c]pyridine-7-carboxylic acid (4-morpholin-4-yl-butyl)-amide (Example 22),
4-Amino-3-(4-bromo-phenoxymethyl)-thieno[3,2-c]pyridine-7-carboxylic acid [2-(2-morpholin-4-yl-ethoxy)-ethyl]-amide (Example 23),
4-Amino-3-(4-bromo-phenoxymethyl)-thieno[3,2-c]pyridine-7-carboxylic acid [4-(4-methoxy-piperidin-1-yl)-butyl]-amide (Example 24),
4-Amino-3-(4-bromo-phenoxymethyl)-thieno[3,2-c]pyridine-7-carboxylic acid [3-(2,3-dihydroxy-propoxy)-propyl]-amide (Example 25).

The compounds of the invention are selective for FGFR and/or KDR kinases. These compounds are useful in the treatment or control of cancer, in particular the treatment or control of solid tumors, specifically breast, lung, colon and prostate tumors. These compounds are highly permeable to cell membranes and thus possess advantageous bioavailability profiles such as improved oral bioavailability.

### General Synthesis of Compounds According to the Invention

The compounds of the present invention can be prepared by any conventional means. Suitable processes for synthesizing these compounds are provided in the examples. Generally, compounds of formula I can be prepared according to the below described synthetic routes. 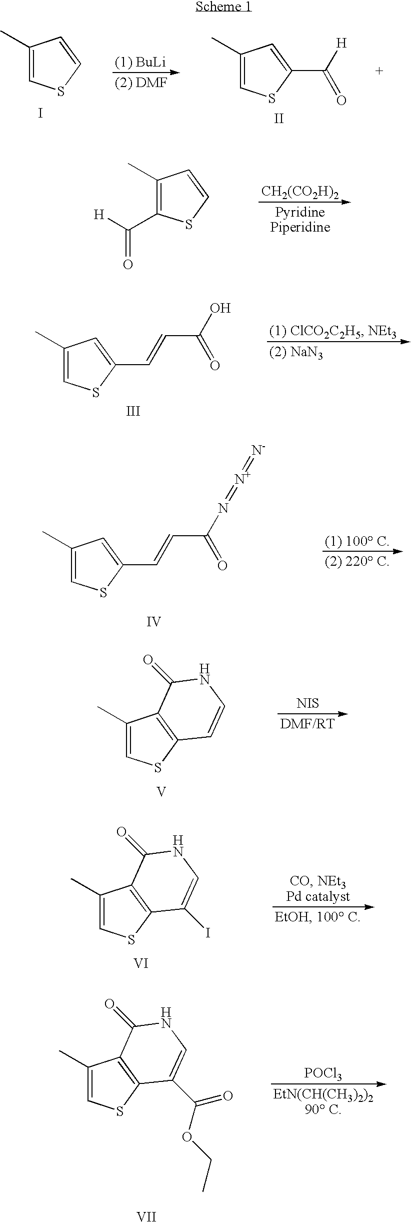 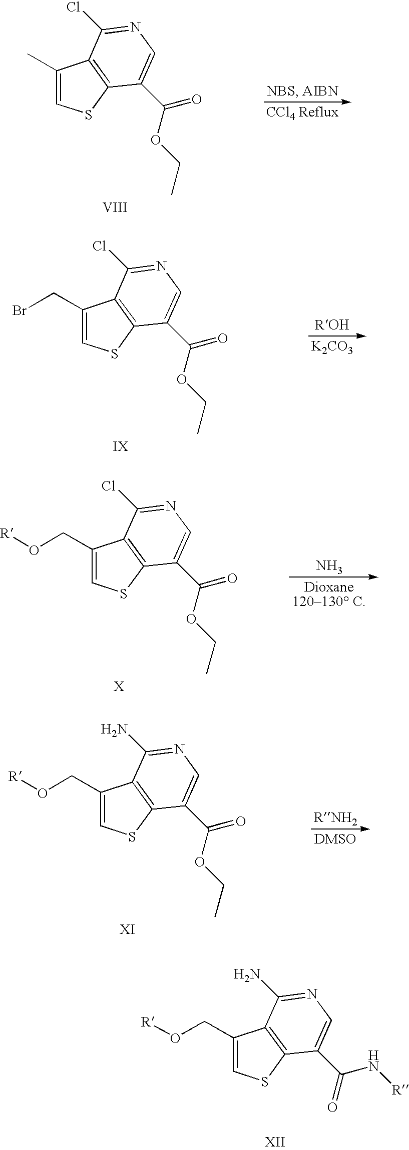 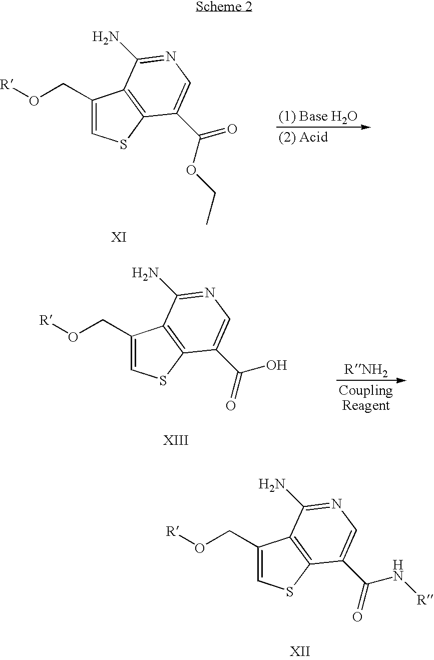
Separating a Mixture of Stereoisomers into the Optically Pure Stereoisomers (When Compound of Formula I is Chiral)

The optional separation of isomeric structures of formula I can be carried out according to known methods such as for example resolution or chiral high pressure liquid chromatography (also known as chiral HPLC). Resolution methods are well known, and are summarized in “Enantiomers, Racemates, and Resolutions” (Jacques, J. et al. John Wiley and Sons, NY, 1981). Methods for chiral HPLC are also well known, and are summarized in “Separation of Enantiomers by Liquid Chromatographic Methods” (Pirkle, W. H. and Finn, J. in “Asymmetric Synthesis”, Vol. 1, Morrison, J. D., Ed., Academic Press, Inc., NY 1983, pp. 87-124).

Converting a Compound of Formula I that Bears a Basic Nitrogen into a Pharmaceutically Acceptable Acid Addition Salt

The optional conversion of a compound of formula I that bears a basic nitrogen into a pharmaceutically acceptable acid addition salt can be effected by conventional means. For example, the compound can be treated with an inorganic acid such as for example hydrochloric acid, hydrobromic acid, sulfuric acid, nitric acid, phosphoric acid, or with an appropriate organic acid such as acetic acid, citric acid, tartaric acid, methanesulfonic acid, p-toluene sulfonic acid, or the like.

Converting a Compound of Formula I that Bears a Carboxylic Acid Group into a Pharmaceutically Acceptable Alkali Metal Salt

The optional conversion of a compound of formula I that bears a carboxylic acid group into a pharmaceutically acceptable alkali metal salt can be effected by conventional means. For example, the compound can be treated with an inorganic base such as lithium hydroxide, sodium hydroxide, potassium hydroxide, or the like.

Converting a Compound of Formula I that Bears a Carboxylic Acid Group or Hydroxy Group into a Pharmaceutically Acceptable Ester

The optional conversion of a compound of formula I that bears a carboxylic acid group or hydroxy group into a pharmaceutically acceptable ester can be effected by conventional means. The conditions for the formation of the ester will depend on the stability of the other functional groups in the molecule to the reaction conditions. If the other moieties in the molecule are stable to acidic conditions, the ester may be conveniently prepared by heating in a solution of a mineral acid (e.g., sulfuric acid) in an alcohol. Other methods of preparing the ester, which may be convenient if the molecule is not stable to acidic conditions include treating the compound with an alcohol in the presence of a coupling agent and in the optional presence of additional agents that may accelerate the reaction. Many such coupling agents are known to one skilled in the art of organic chemistry. Two examples are dicyclohexylcarbodiimide and triphenylphosphine/diethyl azodicarboxylate. In the case where dicyclohexylcarbodiimide is used as the coupling agent, the reaction is conveniently carried out by treating the acid with the alcohol, dicyclohexylcarbodiimide, and the optional presence of a catalytic amount (0-10 mole %) of N,N-dimethylaminopyridine, in an inert solvent such as a halogenated hydrocarbon (e.g., dichloromethane) at a temperature between about 0 degrees and about room temperature, preferably at about room temperature. In the case where triphenylphosphine/diethyl azodicarboxylate is used as the coupling agent, the reaction is conveniently carried out by treating the acid with the alcohol, triphenylphosphine and diethyl azodicarboxylate, in an inert solvent such as an ether (e.g., tetrahydrofuran) or an aromatic hydrocarbon (e.g., toluene) at a temperature between about 0 degrees and about room temperature, preferably at about 0 degrees.

### Compositions/Formulations

In an alternative embodiment, the present invention includes pharmaceutical compositions comprising at least one compound of formula I, or a pharmaceutically acceptable salt or ester thereof and a pharmaceutically acceptable excipient and/or carrier.

These pharmaceutical compositions can be administered orally, for example in the form of tablets, coated tablets, dragees, hard or soft gelatin capsules, solutions, emulsions or suspensions. They can also be administered rectally, for example, in the form of suppositories, or parenterally, for example, in the form of injection solutions.

The pharmaceutical compositions of the present invention comprising compounds of formula I, and/or the salts or esters thereof, may be manufactured in a manner that is known in the art, e.g. by means of conventional mixing, encapsulating, dissolving, granulating, emulsifying, entrapping, dragee-making, or lyophilizing processes. These pharmaceutical preparations can be formulated with therapeutically inert, inorganic or organic carriers. Lactose, corn starch or derivatives thereof, talc, stearic acid or its salts can be used as such carriers for tablets, coated tablets, dragees and hard gelatin capsules. Suitable carriers for soft gelatin capsules include vegetable oils, waxes and fats. Depending on the nature of the active substance, no carriers are generally required in the case of soft gelatin capsules. Suitable carriers for the manufacture of solutions and syrups are water, polyols, saccharose, invert sugar and glucose. Suitable carriers for injection are water, alcohols, polyols, glycerine, vegetable oils, phospholipids and surfactants. Suitable carriers for suppositories are natural or hardened oils, waxes, fats and semi-liquid polyols.

The pharmaceutical preparations can also contain preserving agents, solubilizing agents, stabilizing agents, wetting agents, emulsifying agents, sweetening agents, coloring agents, flavoring agents, salts for varying the osmotic pressure, buffers, coating agents or antioxidants. They can also contain other therapeutically valuable substances, including additional active ingredients other than those of formula I.

### Dosages

As mentioned above, the compounds of the present invention, including the compounds of formula I, are useful in the treatment or control of cell proliferative disorders, including prevention of the formation of new blood vessels in solid tumors (anti-angiogenesis). These compounds and formulations containing said compounds are particularly useful in the treatment or control of solid tumors, such as, for example, breast, colon, lung and prostate tumors.

A therapeutically effective amount of a compound in accordance with this invention means an amount of compound that is effective to prevent, alleviate or ameliorate symptoms of disease or prolong the survival of the subject being treated. Determination of a therapeutically effective amount is within the skill in the art.

The therapeutically effective amount or dosage of a compound according to this invention can vary within wide limits and may be determined in a manner known in the art. Such dosage will be adjusted to the individual requirements in each particular case including the specific compound(s) being administered, the route of administration, the condition being treated, as well as the patient being treated. In general, in the case of oral or parenteral administration to adult humans weighing approximately 70 Kg, a daily dosage of about 10 mg to about 10,000 mg, preferably from about 200 mg to about 1,000 mg, should be appropriate, although the upper limit may be exceeded when indicated. The daily dosage can be administered as a single dose or in divided doses, or for parenteral administration, it may be given as continuous infusion.

### Combinations

The compounds of this invention may be used in combination (administered in combination or sequentially) with known anti-cancer treatments such as radiation therapy or with cytostatic or cytotoxic agents, such as for example, but not limited to, DNA interactive agents, such as cisplatin or doxorubicin; topoisomerase 11 inhibitors such as etoposide: topoisomerase I inhibitors such as CPT-11 or topotecan; tubulin interacting agents, such as paclitaxel, docetaxel or epothilones; hormonal agents such as tamoxifen; thymidilate synthases inhibitors, such as 5-fluorouracil; and anti-metabolites such as methotrexate. Compounds of formula I may also be useful in combination with modulators of p53 transactivation.

If formulated as a fixed dose, the above-described combination products include the compounds of this invention within the dosage range described above and the other pharmaceutically active agent or treatment within its approved dose range. For example, an early cdk1 inhibitor olomucine has been found to act synergistically with well known cytotoxic agents in inducing apoptosis. (J. Cell Sci., 1995, 108, 2897-2904). Compounds of formula I may also be administered sequentially with known anticancer or cytotoxic agents when concomitant administration or a combination is inappropriate. This invention is not limited in the sequence of administration: compounds of formula I may be administered either prior to or after administration of the known anticancer or cytotoxic agent. For example, the cytotoxic activity of the cdk inhibitor flavopiridol is affected by the sequence of administration with anticancer agents. (Cancer Research, 1997, 57, 3375).

The present invention is also directed to the following novel intermediates useful in the synthesis of compounds of formula I:
3-Methyl-5H-thieno[3,2-c]pyridin-4-one (intermediate 4),
7-Iodo-3-methyl-5H-thieno[3,2-c]pyridin-4-one (intermediate 5),
3-Methyl-4-oxo-4,5-dihydro-thieno[3,2-c]pyridine-7-carboxylic acid ethyl ester (intermediate 6),
4-Chloro-3-methyl-thieno[3,2-c]pyridine-7-carboxylic acid ethyl ester (intermediate 7),
3-Bromomethyl-4-chloro-thieno[3,2-c]pyridine-7-carboxylic acid ethyl ester (intermediate 8),
3-(4-Bromo-2,6-difluoro-phenoxymethyl)-4-chloro-thieno[3,2-c]pyridine-7-carboxylic acid ethyl ester (intermediate 9),
4-Amino-3-(4-bromo-2,6-difluoro-phenoxymethyl)-thieno[3,2-c]pyridine-7-carboxylic acid ethyl ester (intermediate 10),
4-Amino-3-(2-chloro-4-methoxy-phenoxymethyl)-thieno[3,2-c]pyridine-7-carboxylic acid ethyl ester (intermediate 11),
3-(4-Bromo-phenoxymethyl)-4-chloro-thieno[3,2-c]pyridine-7-carboxylic acid ethyl ester (intermediate 12),
4-Amino-3-(4-bromo-phenoxymethyl)-thieno[3,2-c]pyridine-7-carboxylic acid ethyl ester (intermediate 13),
4-Chloro-3-phenoxymethyl-thieno[3,2-c]pyridine-7-carboxylic acid ethyl ester (intermediate 14),
4-Amino-3-phenoxymethyl-thieno[3,2-c]pyridine-7-carboxylic acid ethyl ester (intermediate 15), and
4-Amino-3-(4-bromo-phenoxymethyl)-thieno[3,2-c]pyridine-7-carboxylic acid (intermediate 16).

### EXAMPLES

The following examples illustrate preferred methods for synthesizing the compounds and formulations of the present invention.

### Intermediate 1

### 4-Methyl-2-thiophenecarboxaldehyde

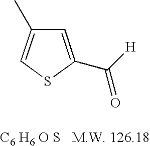

A solution of 3-methylthiophene (58.90 g, 0.60 mol) (Fluka) in anhydrous ether (600 mL) was stirred and cooled in an ice-water bath. This solution was treated dropwise over 15 minutes with n-butyllithium in pentane (2 M, 450 mL, 0.90 mol) (Aldrich). After stirring for 2 hours at room temperature the mixture was cooled in an ice-water bath and treated dropwise over 5 minutes with N,N-dimethylformamide (48.24 g, 0.66 mol) (Fisher) followed by stirring at room temperature over night. The mixture was diluted with ether (600 mL) and washed with water and brine. After drying (sodium sulfate) ether was filtered and evaporated on a rotary evaporator without vacuum to give 114 g of red liquid. This liquid was purified by chromatography over a pad of silica gel 60 (1 Kg, 70-230 mesh) eluting with 40% dichloromethane-hexanes. Evaporation without vacuum gave a mixture of 4-methyl-2-thiophenecarboxaldehyde and 3-methyl-2-thiophenecarboxaldehyde (approximately 5:1) as a light red oil. (Yield 56.62 g, 74.7%).

### Intermediate 2

### 3-(4-Methyl-thiophen-2-yl)-acrylic acid

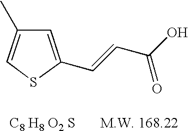

A solution of 4-methyl-2-thiophenecarboxaldehyde (56.62 g, 0.448 mol) (from Intermediate 1 supra, containing 3-methyl-2-thiophenecarboxaldehyde), malonic acid (186.77 g, 1.79 mol) (Aldrich) and piperidine (1.90 g, 0.022 mol) (Fluka) in pyridine (550 mL) was heated at reflux with stirring over night. The reaction mixture was evaporated to dryness. The resulting residue was dissolved in dichloromethane and washed successively with 3 N hydrochloric acid, water and brine. The organic layer was dried (sodium sulfate), filtered, and evaporated to give 3-(4-methyl-thiophen-2-yl)-acrylic acid as a tan solid. (Yield 49.52 g, 65.7%).

### Intermediate 3

### 3-(4-Methyl-thiophen-2-yl)-acryloyl azide

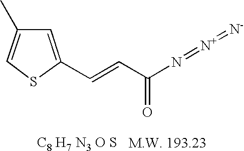

To a solution of 3-(4-methyl-thiophen-2-yl)-acrylic acid (49.52 g, 0.294 mol) (from Intermediate 2 supra) and triethylamine (44.68 g, 0.441 mol) (Aldrich) in acetone (2000 mL) with stirring and cooling in an ice-water bath was added ethyl chloroformate (35.14 g, 0.323 mol) (Aldrich). After stirring at room temperature for 20 minutes, sodium azide (28.70 g, 0.441 mol) (Aldrich) was added and stirring continued for another 20 minutes at room temperature. Acetone was then evaporated off at reduced pressure and residue was diluted with water. This was extracted with dichloromethane. The organic extract was washed with brine, dried (sodium sulfate), filtered, and concentrated to give 3-(4-methyl-thiophen-2-yl)-acryloyl azide as a brown solid. (Yield 48.51 g, 85.4%).

### Intermediate 4

### 3-Methyl-5H-thieno[3,2-c]pyridin-4-one

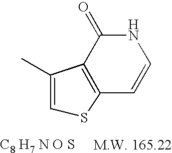

Method A: A mixture of 3-(4-methyl-thiophen-2-yl)-acryloyl azide (69.21 g, 0.358 mol) (from Intermediate 3 supra) and xylene (700 mL) was stirred and heated at reflux for 0.5 hour. Iodine (0.45 g, 1.79 mmol) was added and mixture was heated at reflux over night. Reaction mixture was cooled and stirred for 5 minutes with aqueous sodium bisulfite solution. The suspension was filtered, washed with ether and sucked dry to give 3-methyl-5H-thieno[3,2-c]pyridin-4-one as a tan solid. (Yield 31.28 g, 52.8%).

Method B: 3-(4-Methyl-thiophen-2-yl)-acryloyl azide (1.54 g; 7.95 mmol) (from Intermediate 3 supra) was dissolved in meta-xylenes (16 mL). The solution was heated in an oil bath at 105-115° C. for 30 minutes until nitrogen evolution ceased. At this point a few crystals of iodine were added to the reaction and the oil bath temperature was increased to 145-150° C. The reaction was heated at reflux for 6 hours. Upon cooling, solid precipitated out of solution. Filtration and drying yielded 3-methyl-5H-thieno[3,2-c]pyridine-4-one. (Yield: 1.05 g; 80.1%).

HRMS(EI+) m/z Calcd for C₈H₇NOS [(M⁺)]: 165.0248. Found: 165.0250.

### Intermediate 5

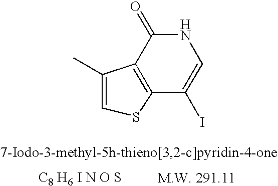

A solution of 3-methyl-5H-thieno[3,2-c]pyridin-4-one (24.27 g, 0.146 mol) (from Intermediate 4 supra) and N-iodosuccinimide (34.70 g, 0.154 mol) (Avocado) in N,N-dimethylformamide (1000 mL) was stirred at room temperature over night. Reaction mixture was concentrated under reduced pressure and residue was stirred with ether (1000 mL) for 0.5 hour. Suspension was filtered, washed with ether and sucked dry to give 7-iodo-3-methyl-5H-thieno[3,2-c]pyridin-4-one as a brown solid. (Yield 41.88 g, 97.9%).

HRMS(EI+) m/z Calcd for C₈H₆INOS [(M⁺)]: 290.9215. Found: 290.9210.

### Intermediate 6

### 3-Methyl-4-oxo-4,5-dihydro-thieno[3,2-c]pyridine-7-carboxylic acid ethyl ester

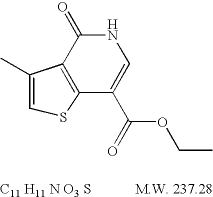

A stirred suspension of 7-iodo-3-methyl-5H-thieno[3,2-c]pyridin-4-one (1.14 g, 3.92 mmol) (from Intermediate 5 supra), triethylamine (2.5 mL, 17.94 mmol) (Aldrich) and bis(triphenylphosphine)palladium(II) chloride (0.14 g, 0.2 mmol) (Aldrich) in ethanol (50 mL) was degassed with argon and then saturated with carbon monoxide. The mixture was stirred with heating in a 75° C. oil bath over night under a blanket of carbon monoxide at atmospheric pressure. After cooling, reaction mixture was concentrated under reduced pressure to remove a portion of ethanol (about 20%). The solid formed was collected by filtration, washed with ethanol-diethyl ether (1:1) and then diethyl ether and finally dried under vacuum to give 3-methyl-4-oxo-4,5-dihydro-thieno[3,2-c]pyridine-7-carboxylic acid ethyl ester. (Yield 0.78 g, 84.0%).

HRMS(EI+) m/z Calcd for C₁₁H₁₁NO₃S [(M⁺)]: 237.0460. Found: 237.0451.

### Intermediate 7

### 4-Chloro-3-methyl-thieno[3,2-c]pyridine-7-carboxylic acid ethyl ester

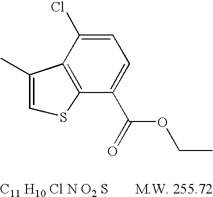

A mixture of 3-methyl-4-oxo-4,5-dihydro-thieno[3,2-c]pyridine-7-carboxylic acid ethyl ester (2.43 g, 10.24 mmol) (from Intermediate 6 supra) and N,N-diisopropylethylamine (2.4 mL, 13.87 mmol) (Fluka) was stirred with cooling in an ice-water bath. This mixture was slowly treated with phosphorous oxychloride (7.8 mL, 83.68 mmol) (Fluka) and then allowed to warm to room temperature. N,N-Dimethylformamide (1.0 mL, 12.86 mmol) was then added and the mixture stirred with heating at 70° C. for 30 minutes. A second portion of N,N-dimethylformamide (0.5 mL, 6.43 mmol) was added and the mixture was heated at 70° C. for another 30 minutes. After cooling, ice was added to the solution and the mixture was extracted with ethyl acetate. The organic extract was washed with water, saturated aqueous sodium bicarbonate solution, water and brine. The aqueous phases were back washed with ethyl acetate. The ethyl acetate solutions were combined, dried (sodium sulfate), filtered, and concentrated under reduced pressure. This residue was purified by flash chromatography over silica gel (Biotage 65M, 5:95 ethyl acetate-hexanes) to give 4-chloro-3-methyl-thieno[3,2-c]pyridine-7-carboxylic acid ethyl ester. (Yield 1.57 g, 60.0%).

HRMS(EI+) m/z Calcd for C₁₁H₁₀ClNO₂S [(M⁺)]: 255.0121. Found: 255.0119.

### Intermediate 8

### 3-Bromomethyl-4-chloro-thieno[3,2-c]pyridine-7-carboxylic acid ethyl ester

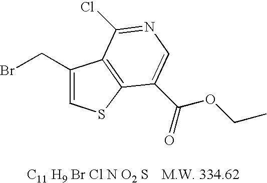

To a solution of 4-chloro-3-methyl-thieno[3,2-c]pyridine-7-carboxylic acid ethyl ester (0.81 g, 3.17 mmol) (from Intermediate 7 supra) in carbon tetrachloride (50 mL) was added N-bromosuccinimide (0.73 g, 4.12 mmol) (Avacado) and 2,2′-azobisisobutyronitrile (52 mg, 0.32 mmol) (Aldrich) respectively. The reaction mixture was heated at 80° C. for 24 h. The mixture was then cooled, concentrated under reduced pressure. The residue was purified by chromatography (ethyl ether-hexanes, 1:4, V/V) to give the desired 3-bromomethyl-4-chloro-thieno[3,2-c]pyridine-7-carboxylic acid ethyl ester as a white solid. (Yield 0.7 g, 66%).

HRMS(EI+) m/z Calcd for C₁₁H₉BrClNO₂S [(M⁺)]: 332.9226. Found: 332.9224.

### Intermediate 9

### 3-(4-Bromo-2,6-difluoro-phenoxymethyl)₄-chloro-thieno[3,2-c]pyridine-7-carboxylic acid ethyl ester

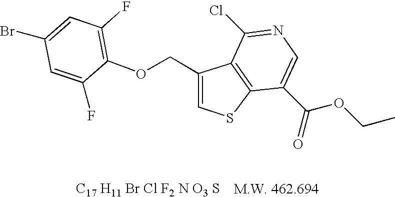

A solution of 3-bromomethyl-4-chloro-thieno[3,2-c]pyridine-7-carboxylic acid ethyl ester (265 mg, 0.79 mmol) (from Intermediate 8 supra) and 2,6-difluoro-4-bromo-phenol (166 mg, 0.79 mmol) (Alfa) in a mixture of tetrahydrofuran-N,N-dimethylformamide (10 mL, 5:1) was treated with potassium carbonate (110 mg, 0.79 mmol). After stirring for 15 hours at room temperature the reaction mixture was warmed to 65° C. and stirred at that temperature for another 5.5 hours. The mixture was then cooled and partitioned between dichloromethane and water. The organic layer was dried over sodium sulfate, filtered and concentrated to a residue that was purified by chromatography with a silica gel column and 0-30% diethyl ether in hexanes to afford the product. Precipitation of this material out of chloroform with excess of hexanes yielded 3-(4-bromo-2,6-difluoro-phenoxymethyl)-4-chloro-thieno[3,2-c]pyridine-7-carboxylic acid ethyl ester as a white solid. (Yield 270 mg, 73%).

HRMS m/z calcd for C₁₇H₁₁BrClF₂NO₃S+H [M+H]⁺: 461.9373. Found: 461.9377.

### Intermediate 10

### 4-Amino-3-(4-bromo-2,6-difluoro-phenoxymethyl)-thieno[3,2-c]pyridine-7-carboxylic acid ethyl ester

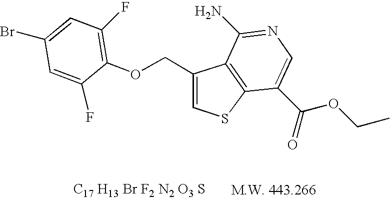

Ammonia gas was bubbled into a solution of 3-(4-bromo-2,6-difluoro-phenoxymethyl)-4-chloro-thieno[3,2-c]pyridine-7-carboxylic acid ethyl ester (200 mg, 0.43 mmol) (from Intermediate 9 supra) in dioxane (10 mL) for 5 minutes in a pressure reactor. The reaction mixture was sealed and stirred at 130° C. for 9 hours and then at room temperature overnight. The solvent was then evaporated under reduced pressure. The resulting residue was purified by silica gel column chromatography with a 0-100% ethyl acetate in hexanes gradient and a precipitation out of tetrahydrofuran with excess of hexanes to give 4-amino-3-(4-bromo-2,6-difluoro-phenoxymethyl)-thieno[3,2-c]pyridine-7-carboxylic acid ethyl ester as a white solid. (Yield 130 mg, 67%).

HRMS m/z calcd for C₁₇H₁₃BrF₂N₂O₃S [M⁺]: 441.9798. Found: 441.9786.

### Intermediate 11

### 4-Amino-3-(2-chloro-4-methoxy-phenoxymethyl)-thieno[3,2-c]pyridine-7-carboxylic acid ethyl ester

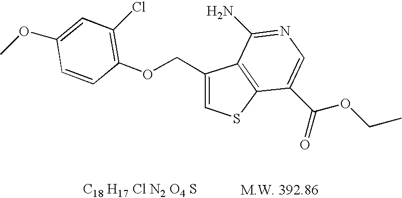

A solution of 3-bromomethyl-4-chloro-thieno[3,2-c]pyridine-7-carboxylic acid ethyl ester (400 mg, 1.19 mmol) (from Intermediate 8 supra) in a mixture of tetrahydrofuran (8 mL) and dichloromethane (2 mL) was added 2-chloro-4-methoxyphenol (192 mg, 1.21 mmol) (Aldrich) and then potassium carbonate (167 mg, 1.21 mmol). Upon consumption of the starting material, as judged by thin layer chromatography, the reaction mixture was partitioned between dichloromethane and water. The organic layer was dried over sodium sulfate, filtered and concentrated. The residue was purified by chromatography with a silica gel column and 0-30% diethyl ether in hexanes gradient to give the intermediate 4-chloro-3-(2-chloro-4-methoxy-phenoxymethyl)-thieno[3,2-c]pyridine-7-carboxylic acid ethyl ester.

Ammonia was bubbled into a solution of this intermediate 4-chloro-3-(2-chloro-4-methoxy-phenoxymethyl)-thieno[3,2-c]pyridine-7-carboxylic acid ethyl ester in dioxane for 5 minutes at room temperature in a pressure reactor. The reaction vessel was then sealed, and the mixture was stirred at 120° C. for 12 hours and at room temperature for 48 hours. The reaction mixture was then evaporated under reduced pressure. The residue was purified by chromatography on a Biotage system with a 20-40% ethyl acetate in hexanes gradient to afford 4-amino-3-(2-chloro-4-methoxy-phenoxymethyl)-thieno[3,2-c]pyridine-7-carboxylic acid ethyl ester as a white powder. (Yield 80 mg, 17%).

HRMS m/z calcd for C₁₈H₁₇ClN₂O₄S [M⁺]: 392.0598. Found: 392.0582.

### Intermediate 12

### 3-(4-Bromo-phenoxymethyl)-4-chloro-thieno[3,2-c]pyridine-7-carboxylic acid ethyl ester

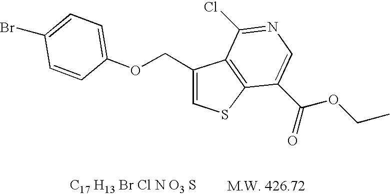

A suspension of potassium carbonate (0.67 g, 4.85 mmol) and 4-bromophenol (0.78 g, 4.47 mmol) (Aldrich) in a tetrahydrofuran-N,N-dimethylformamide mixture (5:1, 40 mL) was heated at 65-70° C. for 3 hours. 3-Bromomethyl-4-chloro-thieno[3,2-c]pyridine-7-carboxylic acid ethyl ester (1.41 g; 4.21 mmol) (from Intermediate 8 supra) was added, rinsing with another portion of tetrahydrofuran-N,N-dimethylformamide solvent mixture (5:1, 13 mL). Heating was continued for 20 hours. The reaction mixture was cooled and concentrated under reduced pressure. The residue was partitioned between dichloromethane and water. The organic phase was washed with water and brine, dried (sodium sulfate), filtered, and concentrated. The crude material was crystallized from hot acetonitrile to give 3-(4-bromo-phenoxymethyl)-4-chloro-thieno[3,2-c]pyridine-7-carboxylic acid ethyl ester. (Yield: 1.15 g, 66.1%).

HRMS(ES+) m/z Calcd for C₁₇H₁₃BrClNO₃S+H [(M+H)⁺]: 425.9561. Found: 425.9562.

### Intermediate 13

### 4-Amino-3-(4-bromo-phenoxymethyl)-thieno[3,2-c]pyridine-7-carboxylic acid ethyl ester

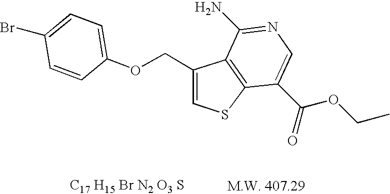

Method A: To a solution of ammonia in dioxane (0.5 N, 200 mL, 100 mmol) (Aldrich) in a pressure tube was added 3-(4-bromo-phenoxymethyl)-4-chloro-thieno[3,2-c]pyridine-7-carboxylic acid ethyl ester (2.1 g, 4.9 mmol) (from Intermediate 12 supra). The reaction mixture was sealed under nitrogen (50 psi) and heated at 100° C. for 48 hour. The mixture was cooled, and concentrated under reduced pressure. The residue was purified by chromatography (ethyl acetate-hexanes, 1:1, then ethyl acetate) to give 4-amino-3-(4-bromo-phenoxymethyl)-thieno[3,2-c]pyridine-7-carboxylic acid ethyl ester as a white solid. (Yield 1.5 g, 75%).

Method B: Ammonia gas was bubbled into a solution of 4-chloro-3-phenoxymethyl-thieno[3,2-c]pyridine-7-carboxylic acid ethyl ester (0.95 g; 2.05 mmol) (from Intermediate 12 supra) in dry dioxane (21 mL) in a pressure bottle for 15 minutes. The bottle was then capped and the solution was heated at 120-125° C. The reaction was monitored by liquid chromatographic analysis and recharged with ammonia after 15 hours. The reaction was stopped after 40 hours. The reaction mixture was concentrated. The residue was partitioned between dichloromethane and water. The organic phase was washed with brine, dried over sodium sulfate and concentrated. The crude mixture was purified by flash chromatography (Biotage 40M; ethyl acetate-hexanes gradient (10-50% ethyl acetate)) to yield 4-amino-3-(4-bromo-phenoxymethyl-thieno[3,2-c]pyridine-7-carboxylic acid ethyl ester. (Yield: 0.65 g, 76.32%).

HRMS(ES+) m/z Calcd for C₁₇H₁₅BrN₂O₃S+H [(M+H)⁺]: 407.0060. Found: 407.0060.

### Intermediate 14

### 4-Chloro-3-phenoxymethyl-thieno[3,2-c]pyridine-7-carboxylic acid ethyl ester

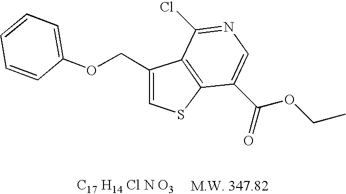

A suspension of potassium carbonate (31 mg; 0.22 mmol) and phenol (22 mg; 0.23 mmol) in tetrahydrofuran-dimethylformamide mixture (2.8 mL, 5:1) was heated at 65° C. for 2 hours. 3-Bromomethyl-3-chloro-thieno[3,2-c]pyridine-7-carboxylic acid ethyl ester (75 mg, 0.22 mmol) (from Intermediate 12 supra) was added, and heating continued overnight. The reaction mixture was cooled and concentrated. The residue was partitioned between dichloromethane and water. The organic phase was washed with brine (2×), dried over sodium sulfate, filtered, and concentrated. The crude material was purified by flash chromatography (Biotage 40S; 75:25 dichloromethane-hexanes) to give 4-chloro-3-phenoxymethyl-thieno[3,2-c]pyridine-7-carboxylic acid ethyl ester. (Yield 19.4 mg, 24.9%).

### Intermediate 15

### 4-Amino-3-phenoxymethyl-thieno[3,2-c]pyridine-7-carboxylic acid ethyl ester

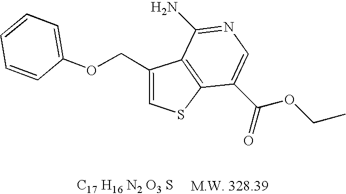

Ammonia gas was bubbled into a solution of 4-chloro-3-phenoxymethyl-thieno[3,2-c]pyridine-7-carboxylic acid ethyl ester (38 mg; 0.11 mmol) (from Intermediate 14 supra) in dioxane (2.4 mL) in a pressure bottle for 30 minutes. The bottle was capped and the clear, colorless solution was heated in an oil bath at 115-125° C. overnight. The crude orange mixture was concentrated and purified by flash chromatography (Biotage 12M; ethyl acetate-hexanes gradient (15-100% ethyl acetate)) to yield 4-amino-3-phenoxymethyl-thieno[3,2-c]pyridine-7-carboxylic acid ethyl ester. (Yield: 14 mg, 39.1%). A significant amount of unreacted 4-chloro-3-phenoxymethyl-thieno[3,2-c]pyridine-7-carboxylic acid ethyl ester (16 mg) was recovered from the chromatography.

### Intermediate 16

### 4-Amino-3-(4-bromo-phenoxymethyl)-thieno[3,2-c]pyridine-7-carboxylic acid

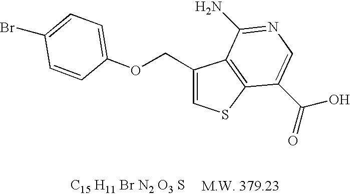

An aqueous solution of sodium hydroxide (1.0 N, 3.1 mL, 3.1 mmol) was added to a solution of 4-amino-3-(4-bromo-phenoxymethyl-thieno[3,2-c]pyridine-7-carboxylic acid ethyl ester (0.75 g; 1.84 mmol) (from Intermediate 13 supra) in tetrahydrofuran-methanol (13 mL, 3:1) and the mixture was heated at 35-40° C. for 18 hours. The crude reaction mixture was concentrated and azeotroped with toluene. The solid residue was triturated with ethyl acetate. The solid was then suspended in water and treated with dilute hydrochloric acid (1.0 N, 3.4 mL). After stirring for 30 minutes, the solid was collected, washed with water and then diethyl ether and dried to yield 4-amino-3-(4-bromo-phenoxymethyl)-thieno[3,2-c]pyridine-7-carboxylic acid. (Yield: 0.67 g, 95.5%).

HRMS(ES+) m/z Calcd for C₁₅H₁₁BrN₂O₃S+H [(M+H)⁺]: 378.9747. Found: 378.9747.

### Intermediate 17

### 4-(4-Aminobutyl)morpholine

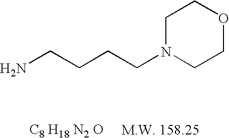

A solution of 4-bromobutylphthalimide (5.0 g, 17.7 mmol) (Lancaster), morpholine (2.0 mL, 23.0 mmol)(Aldrich), and triethylamine (5.0 mL, 35.9 mmol) in absolute ethanol (50 mL) was heated at reflux for 16 hours. Ethanol was removed under reduced pressure. The residue was diluted with dichloromethane and washed with water and brine. After drying (MgSO₄), dichloromethane was evaporated under reduced pressure. The residue was purified by flash chromatography eluting with 4% methanol in dichloromethane to give 2-(4-morpholin-4-yl-butyl)-isoindole-1,3-dione. (Yield 4.44 g, 87%)

To a solution of 2-(4-morpholin-4-yl-butyl)-isoindole-1,3-dione (4.44 g, 15.4 mmol) in absolute ethanol (100 mL) was added hydrazine hydrate (2.0 mL, 41.2 mmol) (Aldrich), and the mixture was heated at reflux for 2 hours. The mixture was then cooled, and filtered, washing the precipitate with absolute ethanol. The combined filtrate and washing was concentrated under reduced pressure. The residue obtained was suspended in dry tetrahydrofuran (100 mL) and cooled in ice. Benzyl chloroformate (Aldrich) (7.5 mL of a 50% solution in toluene, 52.5 mmol) was added dropwise and the mixture was stirred at room temperature for 18 hours. Excess reagent was quenched with methanol. Solvent was removed under reduced pressure. The residue was diluted with water, and the resulting solution was acidified to pH 1 (with dilute hydrochloric acid). This aqueous solution was washed with dichloromethane, then treated with excess sodium carbonate (to pH 10), and extracted with ethyl acetate (3×100 mL). The ethyl acetate layers were combined, dried (MgSO₄), and filtered. Solvent was then removed under reduced pressure and the residue was purified by flash chromatography eluting with a 0-5% methanol in dichloromethane gradient to give N-(benzyloxycarbonyl)-4-(4-aminobutyl)morpholine. (Yield 2.19 g, 49%)

A solution of N-(benzyloxycarbonyl)-4-(4-aminobutyl)morpholine (2.19 g, 7.49 mmol) in methanol (50 mL) was hydrogenated over 10% Pd/C (0.2 g) at 54 psi for 18 hours. The mixture was filtered through a pad of Celite® and concentrated under reduced pressure to give 4-(4-aminobutyl)morpholine which was used without further purification. (Yield 1.43 g, 100%).

### Intermediate 18

### 2-(2-Pyrrolidin-1-yl-ethoxy)-ethylamine

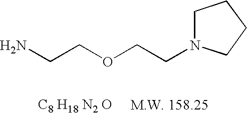

To a solution of 2-(2-aminoethoxy)ethanol (3.5 g, 33.3 mmol) (Aldrich) in dichloromethane (50 mL) at 0° C. was added N-carboethoxyphthalimide (Aldrich) and triethylamine. This mixture was stirred at room temperature for 1 day and then concentrated under reduced pressure. The residue was then purified by flash chromatography eluting with ethyl acetate-hexanes (2:1, V/V) to give 2-[2-(2-hydroxy-ethoxy)-ethyl]-isoindole-1,3-dione. (Yield 3.77 g, 48%)

To a solution of 2-[2-(2-hydroxy-ethoxy)-ethyl]-isoindole-1,3-dione (3.77 g, 16.03 mmol) and carbon tetrabromide (6.38 g, 19.23 mmol) (Aldrich) in dichloromethane (60 mL) at 0° C. was added triphenylphosphine (5.04 g, 19.23 mmol) (Aldrich). The mixture was stirred for 18 hours at room temperature. The reaction mixture was concentrated under reduced pressure and the residue was purified by flash chromatography eluting with ethyl acetate-hexanes (1:2, V/V) to give 2-[2-(2-bromo-ethoxy)-ethyl]-isoindole-1,3-dione. (Yield 4.0 g, 84%).

A solution of 2-[2-(2-bromo-ethoxy)-ethyl]-isoindole-1,3-dione (4.0 g, 13.4 mmol), pyrrolidine (1.46 mL, 17.4 mmol) (Aldrich), and triethylamine (3.74 mL, 26.8 mmol) in absolute ethanol (70 mL) was heated at reflux for 18 hours. Ethanol was removed under reduced pressure. The residue was diluted with dichloromethane and washed with water and brine. After drying (MgSO₄), dichloromethane was evaporated under reduced pressure. The residue was purified by flash chromatography eluting with a 5-10% methanol in dichloromethane gradient to give 2-[2-(2-pyrrolidin-1-yl-ethoxy)-ethyl]-isoindole-1,3-dione. (Yield 1.56 g, 40%).

To a solution of 2-[2-(2-pyrrolidin-1-yl-ethoxy)-ethyl]-isoindole-1,3-dione (1.56 g, 5.41 mmol) in absolute ethanol (20 mL) was added hydrazine hydrate (1.0 mL, 20.6 mmol) (Aldrich). The mixture was heated at reflux for 2 hours, cooled, and filtered, washing the precipitate with absolute ethanol. The filtrate was concentrated and the residue suspended in dry tetrahydrofuran (30 mL) and cooled in ice. Benzyl chloroformate (Aldrich) (2.62 mL of a 50% solution in toluene, 18.39 mmol) was added dropwise. The mixture was stirred at room temperature for 18 hours. Excess reagent was quenched with methanol, and the solvent was removed under reduced pressure. The residue was diluted with water, and the resulting solution was acidified to pH 1 (dilute hydrochloric acid), washed with dichloromethane, then treated with excess sodium carbonate (to pH 10), and extracted with ethyl acetate (3×50 mL). Ethyl acetate layers were combined, dried (MgSO₄), filtered, and concentrated under reduced pressure. This residue was purified by flash chromatography eluting with 0-5% methanol in dichloromethane gradient to give [2-(2-pyrrolidin-1-yl-ethoxy)-ethyl]-carbamic acid benzyl ester. (Yield 1.2 g, 76%).

A solution of [2-(2-pyrrolidin-1-yl-ethoxy)-ethyl]-carbamic acid benzyl ester (1.2 g, 4.1 mmol) in methanol (50 mL) was hydrogenated over 10% Pd/C (0.1 g) at 50 psi for 18 hours. The mixture was filtered through a pad of Celite® and the filtrate was concentrated under reduced pressure to give 2-(2-pyrrolidin-1-yl-ethoxy)-ethylamine which was used without further purification. (Yield 0.85 g, 99%).

### Intermediate 19

### 4-(4-Methoxy-piperidin-1-yl)-butylamine

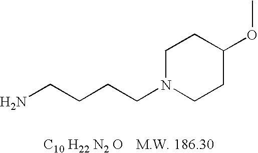

N-Formyl-4-hydroxypiperidine was synthesized from 4-hydroxypiperidine (Aldrich) according to the literature procedure of Baker, W. R. et al. J. Med. Chem., 1992, 35, 1722-1734.

To a solution of N-formyl-4-hydroxypiperidine (10.0 g, 77.4 mmol) in tetrahydrofuran (100 mL) was added sodium hydride (3.41 g, 60% in oil, 85.2 mmol) (Aldrich) at 0° C., followed by stirring for 2 hours at room temperature. The mixture was then re-cooled to 0° C., and iodomethane (5.3 mL, 85.2 mmol) (Aldrich) was added dropwise. The mixture was stirred at room temperature for 18 hours. The reaction was quenched cautiously with water and extracted with ethyl acetate (3×50 mL). Ethyl acetate layers were combined, dried (MgSO₄), and filtered. Solvent was removed under reduced pressure and the residue purified by flash chromatography eluting with 4% methanol in dichloromethane to give 4-methoxy-piperidine-1-carbaldehyde. (Yield 5.63 g, 51%).

A solution of 4-methoxy-piperidine-1-carbaldehyde (5.63 g, 39.30 mmol) and potassium hydroxide (7.37 g, 0.13 mol) in water (40 mL) was stirred at room temperature for 1 day. The reaction mixture was extracted with ether (4×20 mL) and ether layers were combined, dried (MgSO₄) and filtered. This was concentrated to give 4-methoxy-pipeidine which was used without further purification. (Yield 2.43 g, 33%).

A solution of 4-bromobutylphthalimide (5.0 g, 17.7 mmol) (Lancaster), 4-methoxy-pipeidine (2.43 g, 21.3 mmol), and triethylamine (5.0 mL, 35.9 mmol) in absolute ethanol (50 mL) was heated at reflux for 18 hours. Ethanol was removed under reduced pressure. The residue was diluted with dichloromethane and washed with water and brine. After drying (MgSO₄) and filtered, mixture was concentrated under reduced pressure. The residue was purified by flash chromatography eluting with 4% methanol in dichloromethane to give 2-[4-(4-methoxy-piperidin-1-yl)-butyl]-isoindole-1,3-dione. (Yield 4.06 g, 73%).

To a solution of 2-[4-(4-methoxy-piperidin-1-yl)-butyl]-isoindole-1,3-dione (4.06 g, 12.87 mmol) in absolute ethanol (100 mL) was added hydrazine hydrate (2.0 mL, 41.2 mmol) (Aldrich). The reaction mixture was heated at reflux for 2 hours, then cooled, and filtered, washing the precipitate with absolute ethanol. The filtrate was concentrated under reduced pressure and the residue suspended in dry tetrahydrofuran (100 mL) and cooled in ice. Benzyl chloroformate (Aldrich) (6.25 mL of a 50% solution in toluene, 43.77 mmol) was added dropwise followed by stirring at room temperature for 18 hours. Excess reagent was quenched with methanol, and the solvent was removed under reduced pressure. The residue was diluted with water, and the resulting solution was acidified to pH 1 (dilute hydrochloric acid), washed with dichloromethane, then treated with excess sodium carbonate (to pH 10), and extracted with ethyl acetate (3×100 mL). Ethyl acetate layers were combined, dried (MgSO₄), filtered and concentrated under reduced pressure. This residue was purified by flash chromatography eluting with a 5-10% methanol in dichloromethane gradient to give [4-(4-methoxy-piperidin-1-yl)-butyl]-carbamic acid benzyl ester. (Yield 1.9 g, 46%).

A solution of [4-(4-methoxy-piperidin-1-yl)-butyl]-carbamic acid benzyl ester (1.9 g, 5.93 mmol) in methanol (30 mL) was hydrogenated over 10% Pd/C (0.19 g) at 50 psi for 18 hours. The mixture was filtered through a pad of Celite® and the filtrate was concentrated under reduced pressure to give 4-(4-methoxy-piperidin-1-yl)-butylamine which was used without further purification. (Yield 1.43 g, 100%).

### Intermediate 20

### 3-(2,2-Dimethyl-[1,3]dioxolan-4-yl-methoxy)-propylamine

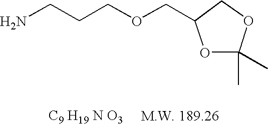

To a solution of 2,2-dimethyl-1,3-dioxolane-4-methanol (26.43 g 0.20 mol) (Aldrich) and acrylonitrile (26.33 mL, 0.40 mol) (Aldrich) in dry tetrahydrofuran (500 mL) at 0° C. was added sodium hydride (1.6 g, 60% in oil, 40 mmol) (Aldrich) slowly. The reaction mixture was stirred at room temperature for 1 hour, then water (100 mL) was added dropwise and the resultant suspension was concentrated under reduced pressure. Water (200 mL) was again added and the mixture was extracted with dichloromethane (2×300 mL). The extracts were combined, dried (MgSO₄), filtered and concentrated to give an oil which was distilled under reduced pressure to give 3-(2,2-dimethyl-[1,3]dioxolan-4-yl-methoxy)-propionitrile. (Yield 26.07 g, 70%; b.p. 86-105° C./0.5 mmHg).

To a solution of 3-(2,2-dimethyl-[1,3]dioxolan-4-yl-methoxy)-propionitrile (13.89 g, 75.0 mmol) in methanol (450 mL) was added cobalt(II) chloride (19.48 g, 0.15 mol) (Aldrich). To this stirred and cooled (ice water bath) solution was added sodium borohydride (28.37 g, 0.75 mol) (Aldrich). Stirring was continued for 1 hour and then concentrated aqueous ammonium hydroxide solution (250 mL) was added. The resultant suspension was filtered and concentrated under reduced pressure to remove methanol. The mixture was extracted with dichloromethane (2×300 mL) and the extracts were combined, dried (MgSO₄) and concentrated under reduced pressure to give an oil which was distilled under reduced pressure to give 3-(2,2-dimethyl-[1,3]dioxolan-4-yl-methoxy)-propylamine. (Yield 7.95 g, 56%; b.p. 75-82° C./0.6 mmHg).

### Example 1

### 4-Amino-3-(4-bromo-2,6-difluoro-phenoxymethyl)-thieno[3,2-c]pyridine-7-carboxylic acid (2-hydroxy-ethyl)-amide

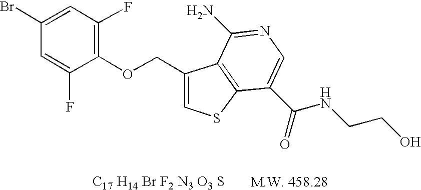

A solution of 4-amino-3-(4-bromo-2,6-difluoro-phenoxymethyl)-thieno[3,2-c]pyridine-7-carboxylic acid ethyl ester (51 mg, 0.11 mmol) (from Intermediate 10 supra) in ethanolamine (approximately 2 mL) (Aldrich) was stirred at 75° C. for 8 hours and at room temperature overnight. This mixture was then partitioned between ethyl acetate and water. The organic layer was dried over sodium sulfate, filtered and concentrated. The residue was purified by silica gel column chromatography with a 0-30% methanol in dichloromethane gradient followed by a precipitation out of tetrahydrofuran with excess of hexanes to give 4-amino-3-(4-bromo-2,6-difluoro-phenoxymethyl)-thieno[3,2-c]pyridine-7-carboxylic acid (2-hydroxy-ethyl)-amide as a white solid. (Yield 22 mg, 42%).

HRMS m/z calcd for C₁₇H₁₄BrF₂N₃O₃S+H [M+H]⁺: 457.9980. Found: 457.9984.

### Example 2

### 4-Amino-3-(2-chloro-4-methoxy-phenoxymethyl)-thieno[3,2-c]pyridine-7-carboxylic acid (2-hydroxy-ethyl)-amide

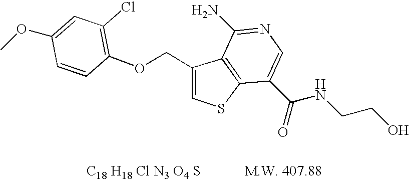

4-Amino-3-(2-chloro-4-methoxy-phenoxymethyl)-thieno[3,2-c]pyridine-7-carboxylic acid ethyl ester (80 mg, 0.20 mmol) (from Intermediate 11 supra) was dissolved in a mixture of ethanolamine (2 mL) (Aldrich) and dimethylsulfoxide (approximately 1 mL). This mixture was stirred at 80° C. overnight and then cooled to room temperature. Water was added and the mixture was filtered to collect the white precipitate that was formed. That precipitate was purified by chromatography with a silica gel column using a 0-10% methanol in dichloromethane gradient. Pure fractions were combined, concentrated and residue was precipitated out of dimethylsulfoxide with excess of water to give 4-amino-3-(2-chloro-4-methoxy-phenoxymethyl)-thieno[3,2-c]pyridine-7-carboxylic acid (2-hydroxy-ethyl)-amide as a white powder. (Yield 20 mg, 25%).

HRMS m/z calcd for C₁₈H₁₈ClN₃O₄S [M⁺]: 407.0707. Found: 407.0700. KDR IC₅₀ 0.5089 μM, FGFR IC₅₀2.559 μM.

### Example 3

### 4-Amino-3-(benzo[1,3]dioxol-5-yloxymethyl)-thieno[3,2-c]pyridine-7-carboxylic acid (2-hydroxy-ethyl)-amide

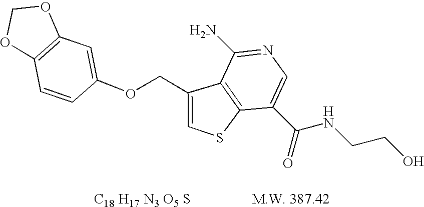

A solution of 3-bromomethyl-4-chloro-thieno[3,2-c]pyridine-7-carboxylic acid ethyl ester (400 mg, 1.19 mmol) (from Intermediate 8 supra) in tetrahydrofuran (8 mL) and dichloromethane (2 mL) was treated with sesamol (167 mg, 1.01 mmol) (Aldrich) and potassium carbonate (167 mg, 1.21 mmol) and stirred at room temperature until thin layer chromatography indicated consumption of the starting material. The reaction mixture was then partitioned between dichloromethane and water. The organic layer was dried over sodium sulfate, filtered and concentrated. The residue was purified with a silica gel column and a 0-30% diethyl ether in hexanes gradient to give the intermediate 3-(benzo[1,3]dioxol-5-yloxymethyl)₄-chloro-thieno[3,2-c]pyridine-7-carboxylic acid ethyl ester.

Ammonia gas was bubbled into a solution of the intermediate 3-(benzo[1,3]dioxol-5-yloxymethyl)₄-chloro-thieno[3,2-c]pyridine-7-carboxylic acid ethyl ester dissolved in dioxane for 5 minutes at room temperature in a pressure tube. The reaction vessel was then sealed, and the mixture was stirred at 120° C. for 12 hours and then at room temperature for 48 hours. Solvent was evaporated off under reduced pressure. The residue resulted was purified by flash chromatography (Biotage system with a 20-40% ethyl acetate in hexanes gradient) to afford 4-amino-3-(benzo[1,3]dioxol-5-yloxymethyl)-thieno[3,2-c]pyridine-7-carboxylic acid ethyl ester that was used in the next step without any further characterization.

A solution of this 4-amino-3-(benzo[1,3]dioxol-5-yloxymethyl)-thieno[3,2-c]pyridine-7-carboxylic acid ethyl ester in ethanolamine (2 mL) (Aldrich) and dimethylsulfoxide (1 mL) was heated in a 120° C. oil bath and stirred until thin layer chromatography indicated consumption of the starting material. The reaction mixture was then cooled and treated with water. The precipitate formed was collected by filtration, washed with water and dried to give 4-amino-3-(benzo[1,3]dioxol-5-yloxymethyl)-thieno[3,2-c]pyridine-7-carboxylic acid (2-hydroxy-ethyl)-amide as a white powder. (Yield 15 mg, 3%).

HRMS m/z calcd for C₁₈H₁₇N₃O₅S [M⁺]: 387.0889. Found: 387.0888.

### Example 4a

### 4-Amino-3-(4-bromo-phenoxymethyl)-thieno[3,2-c]pyridine-7-carboxylic acid (2-hydroxy-ethyl)-amide

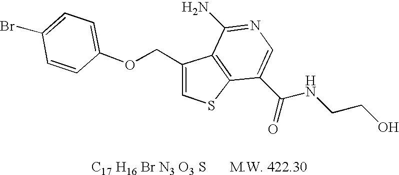

A solution of 4-amino-3-(4-bromophenoxymethyl)-thieno[3,2-c]pyridine-7-carboxylic acid ethyl ester (30 mg, 0.074 mmol) (from Intermediate 13 supra) and ethanolamine (0.50 mL, 8.31 mmol) (Aldrich) in dimethylsulfoxide (0.5 mL) and heated in an oil bath at 70° C. for 10 hours. The reaction was diluted with ethyl acetate and washed with water. The organic phase was concentrated and the residue was treated with water. The solid that formed was collected and was shown to still contain starting material. This solid and the mother liquor was combined with additional 4-amino-3-(4-bromophenoxymethyl)-thieno[3,2-c]pyridine-7-carboxylic acid ethyl ester (14 mg; 0.034 mmol) and dissolved in dimethylsulfoxide (0.5 mL). Ethanolamine (1.0 mL, 16.62 mmol) was added and the mixture heated at 75° C. overnight. The crude reaction was diluted with water, resulting in the precipitation of a milky solid. The addition of ethyl acetate failed to dissolve the solid. The solid was collected and dried to give 4-amino-3-(4-bromo-phenoxymethyl)-thieno[3,2-c]pyridine-7-carboxylic acid (2-hydroxy-ethyl)-amide. (Yield 31.5 mg (90% pure); 62.10%).

A portion of the above material (22 mg) was dissolved in dimethylsulfoxide (0.5 mL) and retreated with ethanolamine (1.0 mL, 16.62 mmol) at 75° C. for 24 hours. The crude reaction was diluted with water and then ethyl acetate, resulting in precipitation of a solid. The solid was collected and dried to give pure 4-amino-3-(4-bromo-phenoxymethyl)-thieno[3,2-c]pyridine-7-carboxylic acid (2-hydroxy-ethyl)-amide. (Yield 20 mg, 43.8% overall).

HRMS(ES+) m/z Calcd for C₁₇H₁₆BrN₃O₃S+H [(M+H)⁺]: 422.0169. Found: 422.0173. KDR IC₅₀ 0.0200 μM, FGFR IC₅₀ 0.0724 μM, VEGF-HUVEC 0.264 μM, FGF-HUVEC 2.762 μM.

### Example 4b

### 4-Amino-3-(4-bromo-phenoxymethyl)-thieno[3,2-c]pyridine-7-carboxylic acid (2-hydroxy-ethyl)-amide bis-methanesulfonic acid salt

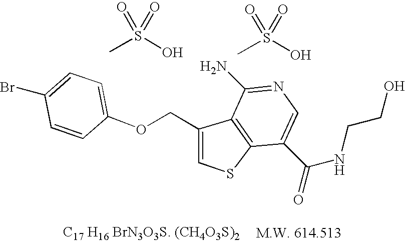

A solution of 4-amino-3-(4-bromo-phenoxymethyl)-thieno[3,2-c]pyridine-7-carboxylic acid (2-hydroxy-ethyl)-amide (0.05 g, 0.12 mmol) (from Example 4a supra) in methanol (5 mL) was treated with methanesulfonic acid (7.7 μL, 0.12 mmol). The mixture was stirred at room temperature for 2 days and then concentrated under reduced pressure. The residue was suspended in water. The solid was filtered and dried to 4-amino-3-(4-bromo-phenoxymethyl)-thieno[3,2-c]pyridine-7-carboxylic acid (2-hydroxy-ethyl)-amide bis-methanesulfonic acid salt as a white powder. (Yield: 25 mg, 34%).

### Example 5

### 4-Amino-3-phenoxymethyl-thieno[3,2-c]pyridine-7-carboxylic acid (2-hydroxy-ethyl)-amide

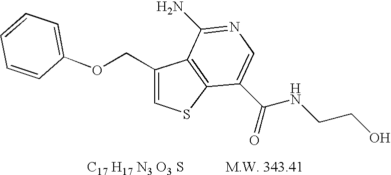

A solution of 4-amino-3-phenoxymethyl-thieno[3,2-c]pyridine-7-carboxylic acid ethyl ester (31 mg; 0.094 mmol) (from Intermediate 15 supra) in dimethylsulfoxide (0.5 mL) was treated with ethanolamine (1.0 mL, 16.62 mmol) (Aldrich) in a pressure bottle and heated at 75° C. for 16 hours. The crude reaction mixture was diluted with water, resulting in the precipitation of a solid. The solid was collected and shown to still contain 15% unreacted starting material. The solid was recombined with the mother liquor and retreated with ethanolamine (1.0 mL) at 75° C. for another 19 hours. The crude reaction mixture was diluted with ethyl acetate and water. The resulting solid was collected, washed with water and diethyl ether and then triturated with acetonitrile to yield 4-amino-3-phenoxymethyl-thieno[3,2-c]pyridine-7-carboxylic acid (2-hydroxy-ethyl)-amide. (Yield 16.7 mg, 51.5%).

HRMS(ES+) m/z Calcd for C₁₇H₁₇N₃O₃S+H [(M+H)⁺]: 344.1064. Found: 344.1066.

### Example 6

### rac-4-Amino-3-(4-bromo-phenoxymethyl)-thieno[3,2-c]pyridine-7-carboxylic acid (2-hydroxy-1-methyl-ethyl)-amide

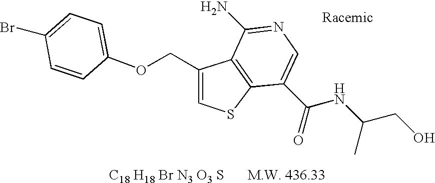

A mixture of 4-amino-3-(4-bromo-phenoxymethyl)-thieno[3,2-c]pyridine-7-carboxylic acid ethyl ester (50 mg, 0.13 mmol) (from Intermediate 13 supra) and racemic 2-amino-1-propanol (2.6 g, 35 mmol) (Aldrich) was heated at 150° C. for 6 hours. The mixture was cooled, diluted with ethyl acetate (50 mL) and then washed with water. The aqueous layer was extracted with ethyl acetate (50 mL). The organic layer was separated, combined and dried over sodium sulfate, filtered, and concentrated under reduced pressure. The residue was purified by chromatography (ethyl acetate-methanol, 85:15) to give rac-4-amino-3-(4-bromo-phenoxymethyl)-thieno[3,2-c]pyridine-7-carboxylic acid (2-hydroxy-1-methyl-ethyl)-amide as a white solid. (Yield 30 mg, 55%).

HRMS m/z calcd for C₁₈H₁₈BrN₃O₃S+H [(M+H)⁺]: 436.0325. Found: 436.0329.

### Example 7

### rac-4-Amino-3-(4-bromo-phenoxymethyl)-thieno[3,2-c]pyridine-7-carboxylic acid (2-hydroxy-propyl)-amide

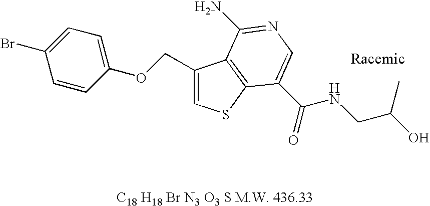

A mixture of 4-amino-3-(4-bromo-phenoxymethyl)-thieno[3,2-c]pyridine-7-carboxylic acid ethyl ester (60 mg, 0.15 mmol) (from Intermediate 13 supra) and racemic 1-amino-2-propanol (3 g, 40 mmol) (Aldrich) was heated at 130° C. for 16 hours. The mixture was cooled, diluted with ethyl acetate (50 mL) and washed with water. The aqueous layer was extracted with ethyl acetate (50 mL). The organic layer was separated, combined and dried over sodium sulfate, filtered and concentrated under reduced pressure. The residue was purified by chromatography (ethyl acetate:methanol, 85:15) to give rac-4-amino-3-(4-bromo-phenoxymethyl)-thieno[3,2-c]pyridine-7-carboxylic acid (2-hydroxy-propyl)-amide as a white solid. (Yield, 34 mg, 52%).

HRMS m/z calcd for C₁₈H₁₈BrN₃O₃S+H [(M+H)⁺]: 436.0325. Found: 436.0329.

### Example 8

### rac-4-Amino-3-(4-bromo-phenoxymethyl)-thieno[3,2-c]pyridine-7-carboxylic acid (2,3-dihydroxy-propyl)-amide

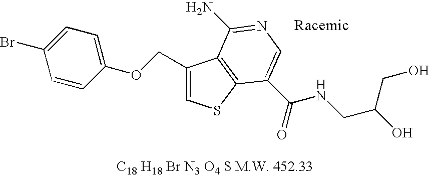

A mixture of 4-amino-3-(4-bromo-phenoxymethyl)-thieno[3,2-c]pyridine-7-carboxylic acid ethyl ester (80 mg, 0.20 mmol) (from Intermediate 13 supra) and racemic 3-amino-1,2-propanediol (3 g, 33 mmol) (Aldrich) was heated at 130° C. for 16 hours. The mixture was cooled, diluted with a co-solvent mixture of ethyl acetate and methanol (1:1, 20 mL). The precipitate formed was filtered, dried and collected to give rac-4-amino-3-(4-bromo-phenoxymethyl)-thieno[3,2-c]pyridine-7-carboxylic acid (2,3-dihydroxy-propyl)-amide as a white solid. (Yield 80 mg, 90%).

HRMS m/z calcd for C₁₈H₁₈BrN₃O₄S+H [(M+H)⁺]: 452.0274. Found: 452.0279.

### Example 9

### 4-Amino-3-(4-bromo-phenoxymethyl)-thieno[3,2-c]pyridine-7-carboxylic acid (2-hydroxy-1,1-dimethyl-ethyl)-amide

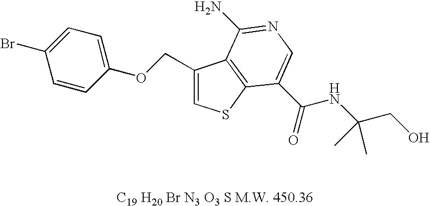

A mixture of 4-amino-3-(4-bromo-phenoxymethyl)-thieno[3,2-c]pyridine-7-carboxylic acid ethyl ester (80 mg, 0.20 mmol) (from Intermediate 13 supra) and 2-amino-2-methyl-1-propanol (4 g, 45 mmol) (Aldrich) was heated at 130° C. for 16 hours. The mixture was cooled, diluted with ethyl acetate (100 mL) and washed with water. The aqueous layer was extracted with ethyl acetate (100 mL). The organic layer was separated, combined and dried over sodium sulfate, filtered, and concentrated under reduced pressure. The residue was purified by chromatography (ethyl acetate:methanol, 10:1) to give 4-amino-3-(4-bromo-phenoxymethyl)-thieno[3,2-c]pyridine-7-carboxylic acid (2-hydroxy-1,1-dimethyl-ethyl)-amide as a white solid. (Yield 30 mg, 33%).

HRMS m/z calcd for C₁₉H₂₀BrN₃O₃S+H [(M+H)⁺]: 450.0482. Found: 450.0487.

### Example 10

### 4-Amino-3-(4-bromo-phenoxymethyl)-thieno[3,2-c]pyridine-7-carboxylic acid (2-hydroxy-1-hydroxymethyl-ethyl)-amide

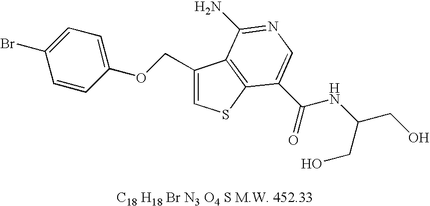

A mixture of 4-amino-3-(4-bromo-phenoxymethyl)-thieno[3,2-c]pyridine-7-carboxylic acid ethyl ester (80 mg, 0.20 mmol) (from Intermediate 13 supra) and 2-amino-1,3-propanediol (3 g, 33 mmol) (Aldrich) was heated at 180° C. for 5 hours. The mixture was cooled, diluted with a co-solvent mixture of ethyl acetate and methanol (1:1, 20 mL). The precipitate formed was filtered, dried and collected to give 4-amino-3-(4-bromo-phenoxymethyl)-thieno[3,2-c]pyridine-7-carboxylic acid (2-hydroxy-1-hydroxymethyl-ethyl)-amide as a white solid. (Yield 60 mg, 66%).

HRMS m/z calcd for C₁₈H₈BrN₃O₄S+H [(M+H)⁺]: 452.0274. Found: 452.0279.

### Example 11

### 4-Amino-3-(4-bromo-phenoxymethyl)-thieno[3,2-c]pyridine-7-carboxylic acid (2-diethylamino-ethyl)-amide

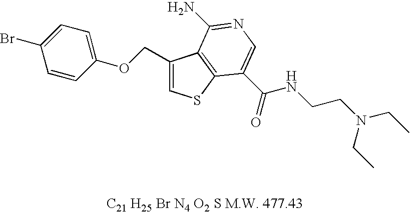

4-Amino-3-(4-bromo-phenoxymethyl)-thieno[3,2-c]pyridine-7-carboxylic acid (20.4 mg, 0.054 mmol) (from Intermediate 16 supra), 1-hydroxy-benzotriazole hydrate (10.0 mg, 0.074 mmol) (Aldrich) and 1,3-diisopropylcarbodiimide (10.0 μL, 0.064 mmol) (Aldrich) were combined in tetrahydrofuran:N,N-dimethylformamide (1.2 mL, 5:1) with vigorous stirring. The reactants briefly went into solution prior to re-precipitation of a solid. The mixture was stirred at room temperature for 30 minutes. N,N-Diethylenediamine (15 μL, 0.11 mmol) (Aldrich) was then added and the mixture was stirred at room temperature overnight. The reaction mixture was concentrated. The residue was taken up in ethyl acetate and washed with water and brine. Any unreacted starting material remained in the aqueous phase. The organic phase was concentrated and purified by reverse-phase chromatography (SB-C18 column, 25 mm×21.2 mm, 5-90% acetonitrile-water (containing 0.75% trifluoroacetic acid) gradient over 10 minutes). The product-containing fractions were combined and freeze-dried. The freeze-dried material (as trifluoroacetic acid salt) was combined with comparable material from another experiment and dissolved in ethyl acetate. The trifluoroacetic acid salt was neutralized by washing with 1N sodium hydroxide and then washing to neutrality with water and brine. The organic phase was dried and concentrated. The residue was recrystallized from ethyl acetate-hexanes to give 4-amino-3-(4-bromo-phenoxymethyl)-thieno[3,2-c]pyridine-7-carboxylic acid (2-diethylamino-ethyl)-amide. (Yield: 32.7 mg, 43.0% combined yield for two experiments).

HRMS(ES+) m/z Calcd for C₂₁H₂₅BrN₄O₂S+H [(M+H)⁺]: 477.0955. Found: 477.0961.

### Example 12a

### 4-Amino-3-(4-bromo-phenoxymethyl)-thieno[3,2-c]pyridine-7-carboxylic acid (4-pyrrolidin-1-yl-butyl)-amide

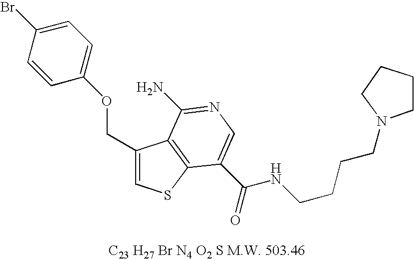

4-Amino-3-(4-bromo-phenoxymethyl)-thieno[3,2-c]pyridine-7-carboxylic acid (21.1 mg, 0.056 mmol) (from Intermediate 16 supra), 1-hydroxy-benzotriazole hydrate (12.0 mg; 0.089 mmol) (Aldrich) and 1,3-diisopropyl-carbodiimide (12.5 μL, 0.080 mmol) (Aldrich) were combined in tetrahydrofuran:N,N-dimethylformamide (1.2 mL, 5:1) with vigorous stirring. The solid slowly went into solution. After 1 hour, 4-pyrrolidinobutylamine (23.0 mg; 0.16 mmol) (Pfaltz & Bauer) was added and stirring continued at room temperature. After approximately 40 hours, the reaction mixture was concentrated. The residue was dissolved in ethyl acetate and washed with water and brine. The organic phase was concentrated and purified by reverse-phase HPLC (SB-C18 column, 25 mm×21.2 mm, 5-90% acetonitrile-water (containing 0.75% trifluoroacetic acid) gradient over 10 minutes), along with material from other reactions. Pure fractions from all runs were combined and freeze-dried. The amorphous solid (trifluoroacetic acid salt) was dissolved in ethyl acetate and neutralized with a 1N sodium hydroxide wash. The organic phase was washed to neutrality with water and brine, dried over sodium sulfate and concentrated. The material was then recrystallized from ethyl acetate-hexanes to give 4-amino-3-(4-bromo-phenoxymethyl)-thieno[3,2-c]pyridine-7-carboxylic acid (4-pyrrolidin-1-yl-butyl)-amide. (Yield 11.9 mg).

HRMS(ES+) m/z Calcd for C₂₃H₂₇BrN₄O₂S+H [(M+H)⁺]: 503.1111. Found: 503.1114.

### Example 12b

### 4-Amino-3-(4-bromo-phenoxymethyl)-thieno[3,2-c]pyridine-7-carboxylic acid (4-pyrrolidin-1-yl-butyl)-amide hydrochloride salt

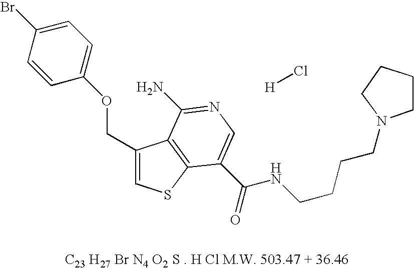

4-Amino-3-(4-bromo-phenoxymethyl)-thieno[3,2-c]pyridine-7-carboxylic acid (0.187 g; 0.49 mmol) (from Intermediate 16 supra), 1-hydroxybenzotriazole hydrate (0.13 g; 0.084 mmol) (Aldrich) and 1,3-diisopropylcarbodiimide (0.11 mL; 0.70 mmol) (Aldrich) were combined in tetrahydrofuran:N,N-dimethylformamide (48 mL, 5:1) with vigorous stirring. The solution was stirred for 4 hours at room temperature, after which time 4-pyrrolidinobutylamine (0.20 g; 1.41 mmol) (Pfaltz & Bauer) was added and stirring continued at room temperature overnight. The reaction was concentrated. The residue was partitioned between ethyl acetate and water. The organic phase was washed with water (2×) and brine and then concentrated. The residue was dissolved in aqueous trifluoroacetic acid, filtered to remove insoluble material and then freeze-dried. The freeze-dried trifluoroacetic acid salt was diluted with ethyl acetate, neutralized with 1N sodium hydroxide to form the free base and then washed with water and brine. The organic phase was concentrated. The free base residue was dissolved in hot tetrahydrofuran (30 mL) and treated with 1 equivalent of aqueous 1 N hydrochloric acid. The resulting hydrochloride salt precipitated out of solution. The solid was collected, redissolved in water and freeze-dried to give 4-amino-3-(4-bromo-phenoxymethyl)-thieno[3,2-c]pyridine-7-carboxylic acid (4-pyrrolidin-1-yl-butyl)-amide hydrochloride. (Yield 0.17 g, 65.7%).

HRMS(ES+) m/z Calcd for C₂₃H₂₇BrN₄O₂S+H [(M+H)⁺]: 503.1111. Found: 503.1105.

### Example 12c

### 4-Amino-3-(4-bromo-phenoxymethyl)-thieno[3,2-c]pyridine-7-carboxylic acid (4-pyrrolidin-1-yl-butyl)-amide methanesulfonic acid salt

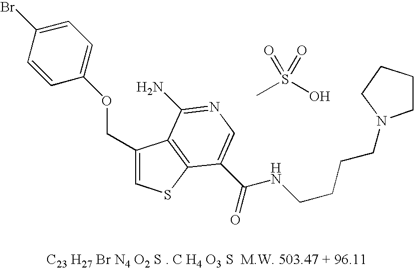

This compound may be prepared in a manner analogous to the compound of Example 12b using the corresponding amide and methanesulfonic acid.

### Example 13a

### 4-Amino-3-(4-bromo-phenoxymethyl)-thieno[3,2-c]pyridine-7-carboxylic acid (2-morpholin-4-yl-ethyl)-amide

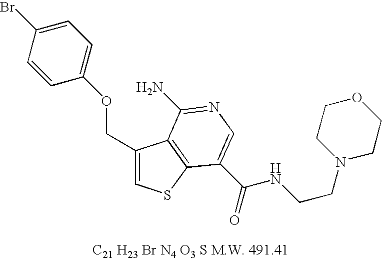

N-(2-Aminoethyl)-morpholine (2.0 mL) (Aldrich) and methanol (2.0 mL) were combined and stirred over sodium sulfate and basic alumina for 2-3 hours. A portion of this solution (1.0 mL; 3.8 mmol) was added to a mixture of 4-amino-3-(4-bromo-phenoxymethyl)-thieno[3,2-c]pyridine-7-carboxylic acid ethyl ester (36.3 mg; 0.089 mmol) (from Intermediate 13 supra) and sodium cyanide (12.0 mg; 0.25 mmol). A clear solution quickly was obtained and the solution was heated at 65° C. Solid began to precipitate out of solution after 3 hours. Liquid chromatographic analysis of the reaction after 42 hours showed about 1:1 mixture of the desired product and the acid by-product (4-amino-3-(4-bromo-phenoxymethyl)-thieno[3,2-c]pyridine-7-carboxylic acid). The reaction was diluted with ethyl acetate. The organic solution was washed with water and brine, dried over sodium sulfate and concentrated. The residue was recrystallized from ethyl acetate-hexanes to yield 4-amino-3-(4-bromo-phenoxymethyl-thieno[3,2-c]pyridine-7-carboxylic acid (2-morpholin-4-yl-ethyl)-amide. (Yield 11.0 mg, 25.1%).

HRMS(ES+) m/z Calcd for C₂₁H₂₃BrN₄O₃S+H [(M+H)⁺]: 491.0747. Found: 491.0749.

### Example 13b

### 4-Amino-3-(4-bromo-phenoxymethyl)-thieno[3,2-c]pyridine-7-carboxylic acid (2-morpholin-4-yl-ethyl)-amide hydrochloride salt

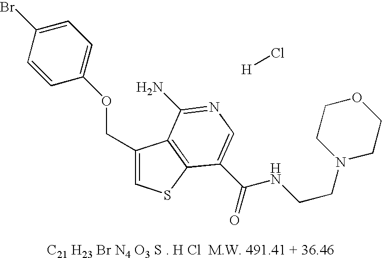

4-Amino-3-(4-bromo-phenoxymethyl)-thieno[3,2-c]pyridine-7-carboxylic acid (40.7 mg, 0.11 mmol) (from Intermediate 16 supra), 1-hydroxybenzotriazole hydrate (23.8 mg, 0.18 mmol) (Aldrich) and 1,3-diisopropylcarbodiimide (25 μL; 0.16 mmol) (Aldrich) were combined in tetrahydrofuran:N,N-dimethylformamide (16 mL, 5:1) with vigorous stirring. The solution was stirred for 3.75 hours at room temperature after which time, N-(2-aminoethyl)-morpholine (42 μL; 0.32 mmol) (source) was added and stirring continued at room temperature. The reaction was concentrated after 40 hours. The residue was taken up in ethyl acetate and the resulting organic phase was washed with water and brine. The organic phase was concentrated. The residue was dissolved in aqueous trifluoroacetic acid, filtered to remove insoluble material and purified by reverse-phase HPLC (SB-C18 column, 25 mm×21.2 mm, 5-90% acetonitrile-water (containing 0.75% trifluoroacetic acid) gradient over 10 minutes) in multiple runs. The pure product-containing fractions were combined and concentrated to near dryness. The residue was diluted with ethyl acetate, neutralized with 1N sodium hydroxide to form the free base and then washed with water and brine. The free base (27.4 mg; 0.056 mmol) was dissolved in hot tetrahydrofuran and treated with 1 equivalent of aqueous 1N hydrochloric acid (53 μL). The resulting hydrochloride salt precipitated out of solution. The solid was collected and dried to give 4-amino-3-(4-bromo-phenoxymethyl-thieno[3,2-c]pyridine-7-carboxylic acid (2-morpholin-4-yl-ethyl)-amide hydrochloride. (Yield 16.2 mg; 28.6%).

HRMS(ES+) m/z Calcd for C₂₁H₂₃BrN₄O₃S+H [(M+H)⁺]: 491.0747. Found: 491.0746. KDR IC₅₀ 0.0584 μM, FGFR IC₅₀ 0.1803 μM, VEGF-HUVEC 0.204 μM, FGF-HUVEC 0.627 μM.

### Example 14

### 4-Amino-3-(4-bromo-phenoxymethyl)-thieno[3,2-c]pyridine-7-carboxylic acid (3-dimethylamino-2,2-dimethyl-propyl)-amide

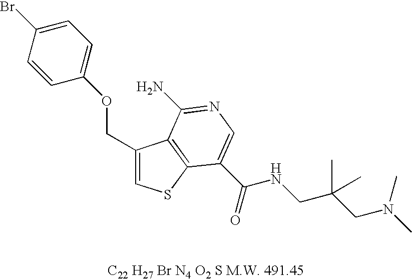

To a solution of 4-amino-3-(4-bromo-phenoxymethyl)-thieno[3,2-c]pyridine-7-carboxylic acid (0.1 g, 0.26 mmol) (from Intermediate 16 supra) and N,N,2,2-tetramethyl-1,3-propanediamine (3 equiv, 0.1 g, 0.79 mmol) (Aldrich) in anhydrous N,N-dimethylformamide and acetonitrile (1:1, 10 mL) was added diphenylphosphoryl azide (0.29 g, 1.06 mmol) (Aldrich) and triethylamine (3 equiv, 0.08 g, 0.79 mmol) (Aldrich). The reaction mixture was stirred at room temperature for 16 hours then diluted with ethyl acetate (100 mL), and washed with water. The organic layer was separated, dried over sodium sulfate, filtered, and concentrated under reduced pressure. The residue was purified by chromatography (ethyl acetate:methanol:triethylamine, 9:1:0.04) to give 4-amino-3-(4-bromo-phenoxymethyl)-thieno[3,2-c]pyridine-7-carboxylic acid (3-dimethylamino-2,2-dimethyl-propyl)-amide as a white solid. (Yield 24 mg, 19%).

HRMS m/z calcd for C₂₂H₂₇BrN₄O₂S+CH₃OH+H [(M+CH₃OH+H)⁺]: 523.1373. Found: 523.1347.

### Example 15

### rac-4-Amino-3-(4-bromo-phenoxymethyl)-thieno[3,2-c]pyridine-7-carboxylic acid (5-diethylamino-1-methyl-pentyl)-amide

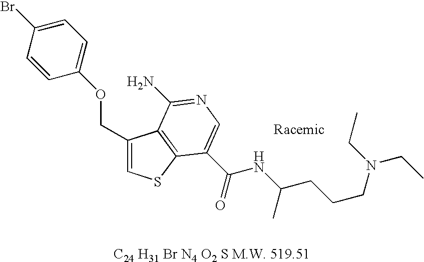

To a solution of 4-amino-3-(4-bromo-phenoxymethyl)-thieno[3,2-c]pyridine-7-carboxylic acid (0.1 g, 0.26 mmol) (from Intermediate 16 supra) and racemic 2-amino-5-diethylaminopentane (0.12 g, 0.79 mmol) (Aldrich) in anhydrous N,N-dimethylformamide and acetonitrile (1:1, 10 mL) was added diphenylphosphoryl azide (0.29 g, 1.06 mmol) (Aldrich) and triethylamine (0.08 g, 0.79 mmol). The reaction mixture was stirred at room temperature for 16 hours, then diluted with ethyl acetate (100 mL), and washed with water. The organic layer was separated, dried over sodium sulfate, and concentrated under reduced pressure. The residue was purified by chromatography (ethyl acetate:methanol:triethylamine, 8:2:0.04) to give rac-4-amino-3-(4-bromo-phenoxymethyl)-thieno[3,2-c]pyridine-7-carboxylic acid (4-diethylamino-1-methyl-butyl)-amide as a white solid. (Yield 21 mg, 16%).

HRMS m/z calcd for C₂₄H₃₁BrN₄O₂S+H [(M+H)⁺]: 519.1424. Found: 519.1426.

### Example 16

### 4-Amino-3-(4-bromo-phenoxymethyl)-thieno[3,2-c]pyridine-7-carboxylic acid amide

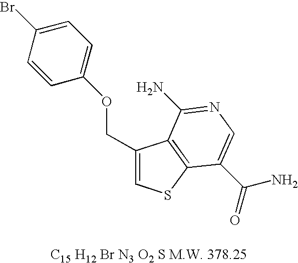

To a solution of 4-amino-3-(4-bromo-phenoxymethyl)-thieno[3,2-c]pyridine-7-carboxylic acid (0.1 g, 0.26 mmol) (from Intermediate 16 supra) in thionyl chloride (20 mL) was added a drop of triethylamine (0.1 mL). The reaction mixture was then heated at 80° C. for 1 hour. The reaction mixture was cooled and concentrated to dryness. To the residue was added a methanolic solution of ammonia (20 mL, 40 mmol, 2 N). The reaction mixture was then stirred at room temperature for 24 hours. The mixture was concentrated, and the residue was purified by chromatography (ethyl acetate:methanol, 20:1) to give 4-amino-3-(4-bromo-phenoxymethyl)-thieno[3,2-c]pyridine-7-carboxylic acid amide as an off white solid. (Yield 35 mg, 36%).

HRMS m/z calcd for C₁₅H₁₂BrN₃O₂S—H₂ [(M−2H)+]: 376.9834. Found: 376.9813.

The compounds of Examples 17-24 unless specifically exemplified can be prepared in a manner analogous to the compounds of Examples 12a and 12b using the corresponding amines to form the appropriate compounds.

### Example 17

### 4-Amino-3-(4-bromo-phenoxymethyl)-thieno[3,2-c]pyridine-7-carboxylic acid [2-(2-pyrrolidin-1-yl-ethoxy)-ethyl]-amide

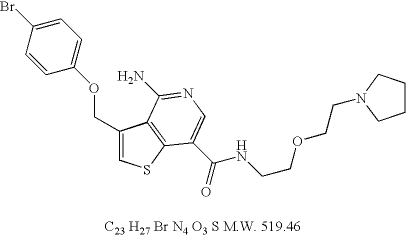

4-Amino-3-(4-bromo-phenoxymethyl)-thieno[3,2-c]pyridine-7-carboxylic acid (0.05 g, 0.13 mmol) (from Intermediate 16 supra), 1-hydroxybenzotriazole hydrate (28.5 mg, 0.21 mmol) (Aldrich) and 1,3-diisopropylcarbodiimide (0.03 mL, 0.19 mmol) (Aldrich) were combined in a mixture of tetrahydrofuran-N,N-dimethylformamide (3.6 mL, 5:1, V/V) with stirring. After 1 hour, 2-(2-pyrrolidin-1-yl-ethoxy)-ethylamine (0.06 g, 0.40 mmol) (from Intermediate 18 supra) was added. The mixture was stirred at room temperature for 3 days and then concentrated under reduced pressure. The residue was purified by C18 column chromatography eluting with acetonitrile-water (20-90% gradient) to give 4-amino-3-(4-bromo-phenoxymethyl)-thieno[3,2-c]pyridine-7-carboxylic acid [2-(2-piperidin-1-yl-ethoxy)-ethyl]-amide as a white powder. (Yield 36.6 mg, 54%).

HRMS (ES⁺) m/z Calcd for C₂₃H₂₇BrN₄O₃S+H [(M+H)⁺]: 519.1060. Found: 519.1060.

### Example 18

### 4-Amino-3-(4-bromo-phenoxymethyl)-thieno[3,2-c]pyridine-7-carboxylic acid [4-(3-methoxy-pyrrolidin-1-yl)-butyl]-amide

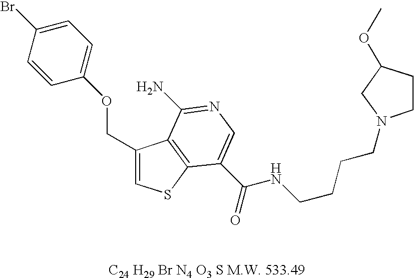

### Example 19

### 4-Amino-3-(4-bromo-phenoxymethyl)-thieno[3,2-c]pyridine-7-carboxylic acid (4-piperidin-1-yl-butyl)-amide

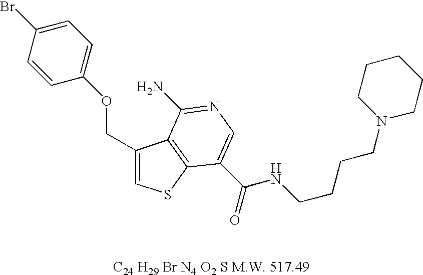

### Example 20

### 4-Amino-3-(4-bromo-phenoxymethyl)-thieno[3,2-c]pyridine-7-carboxylic acid [2-(2-piperidin-1-yl-ethoxy)-ethyl]-amide

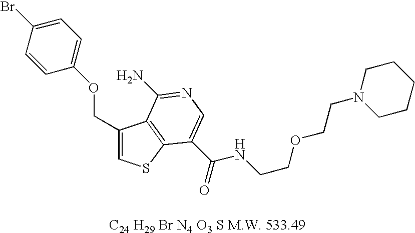

### Example 21

### 4-Amino-3-(4-bromo-phenoxymethyl)-thieno[3,2-c]pyridine-7-carboxylic acid [4-(3-methoxy-piperidin-1-yl)-butyl]-amide

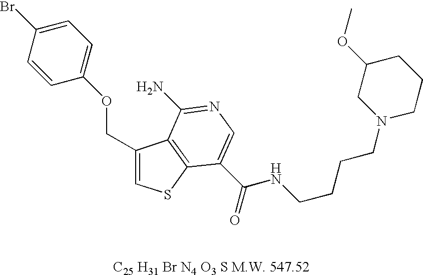

### Example 22

### 4-Amino-3-(4-bromo-phenoxymethyl)-thieno[3,2-c]pyridine-7-carboxylic acid (4-morpholin-4-yl-butyl)-amide

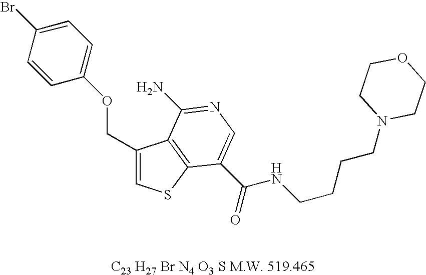

To a solution of 4-amino-3-(4-bromo-phenoxymethyl)-thieno[3,2-c]pyridine-7-carboxylic acid (0.05 g, 0.13 mmol) (from Intermediate 16 supra) in thionyl chloride (20 mL) (Aldrich) was added a drop of triethylamine (0.1 mL). The reaction mixture was then heated at 80° C. for 1 hour. The reaction mixture was cooled and concentrated under reduced pressure to dryness. To the residue in dry tetrahydrofuran (15 mL) was added 4-(4-aminobutyl)morpholine (0.06 g, 0.40 mmol) (from Intermediate 17 supra). The mixture was stirred at room temperature for 18 hours and then concentrated under reduced pressure. The residue was purified by C18 column chromatography eluting with acetonitrile-water (40-80% gradient) to give 4-amino-3-(4-bromo-phenoxymethyl)-thieno[3,2-c]pyridine-7-carboxylic acid (4-morpholin-4-yl-butyl)-amide. (Yield 34 mg, 51%).

HRMS (ES⁺) m/z Calcd for C₂₃H₂₇BrN₄O₃S+H [(M+H)⁺]: 519.1060. Found: 519.1060.

### Example 23

### 4-Amino-3-(4-bromo-phenoxymethyl)-thieno[3,2-c]pyridine-7-carboxylic acid [2-(2-morpholin-4-yl-ethoxy)-ethyl]-amide

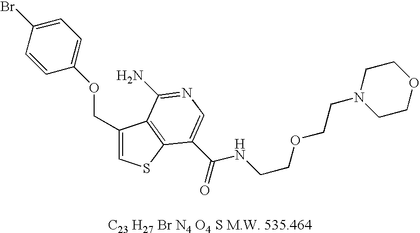

### Example 24

### 4-Amino-3-(4-bromo-phenoxymethyl)-thieno[3,2-c]pyridine-7-carboxylic acid [4-(4-methoxy-piperidin-1-yl)-butyl]-amide

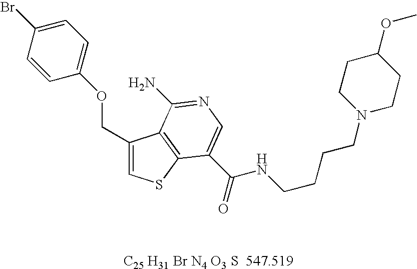

4-Amino-3-(4-bromo-phenoxymethyl)-thieno[3,2-c]pyridine-7-carboxylic acid (0.05 g, 0.13 mmol) (from Intermediate 16 supra), 1-hydroxybenzotriazole hydrate (28.5 mg, 0.21 mmol) (Aldrich) and 1,3-diisopropylcarbodiimide (0.03 mL, 0.19 mmol) (Aldrich) were combined in a mixture of tetrahydrofuran-N,N-dimethylformamide (3.6 mL, 5:1, V/V) with stirring. After 1 hour, 4-(4-methoxy-piperidin-1-yl)-butylamine (0.07 g, 0.40 mmol) (from Intermediate 19 supra) was added. The mixture was stirred at room temperature for 3 days and then concentrated under reduced pressure. The residue was purified by C18 column chromatography eluting with acetonitrile-water (20-90% gradient) to give 4-amino-3-(4-bromo-phenoxymethyl)-thieno[3,2-c]pyridine-7-carboxylic acid [2-(2-piperidin-1-yl-ethoxy)-ethyl]-amide as a white powder. (Yield 22.0 mg, 31%).

HRMS (ES⁺) m/z Calcd for C₂₅H₃₁BrN₄O₃S+H [(M+H)⁺]: 547.1373. Found: 547.1372.

### Example 25

### 4-Amino-3-(4-bromo-phenoxymethyl)-thieno[3,2-c]pyridine-7-carboxylic acid [3-(2,3-dihydroxy-propoxy)-propyl]-amide

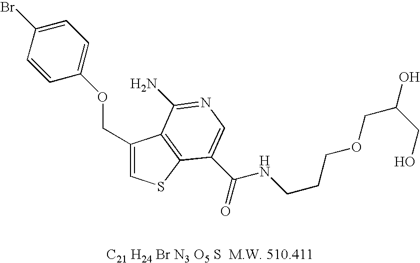

4-Amino-3-(4-bromo-phenoxymethyl)-thieno[3,2-c]pyridine-7-carboxylic acid (0.05 g, 0.13 mmol) (from Intermediate 16 supra), 1-hydroxybenzotriazole hydrate (28.5 mg, 0.21 mmol) (Aldrich) and 1,3-diisopropylcarbodiimide (0.03 mL, 0.19 mmol) (Aldrich) were combined in a mixture of tetrahydrofuran-N,N-dimethylformamide (3.6 mL, 5:1, V/V) with stirring. After 1 hour, 3-(2,2-dimethyl-[1,3]dioxolan-4-yl-methoxy)-propylamine (75.7 mg, 0.40 mmol) (from Intermediate 20 supra) was added. The mixture was stirred at room temperature for 18 hours and then concentrated under reduced pressure. The residue was diluted with ethyl acetate and washed with water and brine, dried (MgSO₄), filtered, and concentrated under reduced pressure. This residue was purified by flash chromatography eluting with hexanes-ethyl acetate (80-100% gradient) to give 4-amino-3-(4-bromo-phenoxymethyl)-thieno[3,2-c]pyridine-7-carboxylic acid [3-(2,2-dimethyl-[1,3]dioxolan-4-ylmethoxy)-propyl]-amide. (Yield 60 mg, 84%).

To a solution of 4-amino-3-(4-bromo-phenoxymethyl)-thieno[3,2-c]pyridine-7-carboxylic acid [3-(2,2-dimethyl-[1,3]dioxolan-4-ylmethoxy)-propyl]-amide (60.0 mg, 0.11 mmol) in ethanol (2 mL) was added 1N aqueous hydrochloric acid (2 mL). The mixture was stirred at room temperature for 18 hours. Mixture was then cooled in an ice-water bath and aqueous 1N sodium hydroxide solution (2 mL) was added. Ethanol was then removed under reduce pressure. The residue was washed with water and purified by flash chromatography eluting with 10% methanol in ethyl acetate to give rac-4-amino-3-(4-bromo-phenoxymethyl)-thieno[3,2-c]pyridine-7-carboxylic acid [3-(2,3-dihydroxy-propoxy)-propyl]-amide as a white powder. (Yield 32 mg, 57%).

HRMS (ES⁺) m/z Calcd for C₂₁H₂₄BrN₃O₅S+H [(M+H)⁺]: 510.0693. Found: 510.0693.

### Antiproliferative Activity

The antiproliferative activity of the compounds of the invention is demonstrated below in Examples 26 and 27. These activities indicate that the compounds of the present invention are useful in treating cancer, in particular solid tumors, more particularly cancerous solid tumors of the breast, lung, prostate and colon, most particularly cancerous solid tumors of the breast and colon.

### Example 26

### Kinase Assay

To determine inhibition of KDR and FGFR, kinase assays were conducted using an HTRF (Homogeneous Time Resolved Fluorescence) assay. This assay is described in A. J. Kolb et. al., Drug Discovery Today, 1998, 3(7), p 333.

Prior to kinase reaction, recombinant EEE-tagged KDR was activated in the presence of activation buffer (50 mM HEPES, pH 7.4, 1 mM DTT, 10% glycerol, 150 mM NaCl, 0.1 mM EDTA, 26 mM MgCl₂, and 4 mM ATP). The enzyme was incubated at 4° C. for 1 hour.

Kinase activity assays were performed in 96-well polypropylene plates (Falcon) with a total volume of 90 μL in each well. Each well contained 1 μM KDR substrate (Biotin-EEEEYFELVAKKKK), 1 nM activated KDR, and a test compound with one of 8 assay concentrations ranging from 100 μM to 128 pM (1:5 serial dilution). The kinase activity assay was done in the presence of 100 mM HEPES, pH 7.4, 1 mM DTT, 0.1 mM Na₂VO₄, 25 mM MgCl₂, 50 mM NaCl (from KDR stock solution), 1% DMSO (from compound), 0.3 mM ATP (at Kₘ concentration) and 0.02% BSA. The reaction was incubated at 37° C. for 30 minutes. To stop the KDR reaction, 72 μL of reaction mixture was transferred into a STOP plate containing 18 μL of revelation buffer (20 mM EDTA, 50 mM HEPES, pH 7.4, 0.02% BSA, 10 nM Eu-labelled anti-pY antibody (final conc. 2 nM), and 100 nM streptavidin (final conc. 20 nM)). After mixing, 35 μL of solution was transferred into duplicate wells of a 384-well black plate (Costar), and read at 615/665 nm on a Wallac Victor 5 reader.

FGFR activity assays were carried out as described above for the KDR activity assay with the following differences. GST-tagged FGFR enzyme was activated at room temperature for 1 hour in the following activation buffer: 100 mM HEPES, pH 7.4, 50 mM NaCl, 20 mM MgCl₂, and 4 mM ATP. The kinase activity assay was performed with 1 μM substrate (Biotin-EEEEYFELV), 1.5 nM activated FGFR, and test compound in the presence of 100 mM HEPES, 1 mM DTT, 0.4 mM MgCl₂, 0.4 mM MnCl₂, 50 mM NaCl, 1% DMSO, 10 μM ATP (Kₘ=8.5 μM for FGFR), 0.1 mM Na₂VO₄, and 0.02% BSA, in a total volume of 90 μL. The rest of the assay was performed in the same manner as KDR assay.

Compound IC₅₀ values were determined from duplicate sets of data, and calculated by using Excel and fitting data to equation Y=[(a−b)/{1+(X/c)^{d}]+b, where a and b are enzyme activity in the presence of no test inhibitor compound and an infinite amount of inhibitor test compound, respectively, c is the IC₅₀ and d is the hill constant of the compound response. The IC₅₀ value is the concentration of test compound that reduces by 50% the enzyme activity under the test conditions described.

The compounds of the present invention have KDR IC₅₀ values less than 5 μM, preferably less than 1.5 μM, or FGFR IC₅₀ values less than 5 μM, preferably less than 2.5 μM. Most preferably, the compounds of the invention have KDR IC₅₀ values less than 1.5 μM and FGFR IC₅₀ values less than 2.5 μM.

### Example 27

### VEGF and FGF-Stimulated HUVEC Proliferation Assays

The antiproliferative activity of test compounds of this invention in cell-based assays was evaluated by BrdU assay using the BrdU kit (Roche Biochemicals 1-647-229). Human umbilical vein endothelial cells (HUVEC, Clonetics CC-2519) were cultured in EGM-2 (Clonetics CC-3162) medium and seeded at 10000 cells per well in a volume of 200 μL of EGM-2 (Clonetics CC-3162) media in a 96-well flat bottom plates (Costar 3595) overnight. After 24 hours of growth at 37° C. with 5% CO₂, the incubation media was removed slowly by aspiration and the content of each well was washed with 300 μL pre-warmed EBM-2 (Clonetics CC-3156) containing 50 μg per mL of gentamycin and 50 ng per mL of amphotercin-B (Clonetics CC-4083). Subsequently, the remaining media was again aspirated and replaced with 160 μL per well of serum starvation media (EBM-2 supplemented with 1% heat inactivated FBS (Clonetics CC-4102), 50 μg per mL gentamycin and 50 ng per mL of amphotercin-B (Clonetics CC-4083), 10 units per mL of Wyeth-Ayerst heparin (NDC0641-0391-25), and 2 mM L-glutamine (GIBCO 25030-081). After serum starving the cells for 24 hours, 20 μL of test compound at 10× test concentration in serum starvation medium with 2.5% DMSO was added to the appropriate wells. The control wells contained 20 μL of serum starvation medium with 2.5% DMSO. Plates were returned to the incubator for 2 hours. After pre-incubating the cells with the test compounds for 2 hours, 20 μL of growth factors at 10× assay concentration diluted in serum starvation media, FGF at 50 ng per mL, or VEGF (R&D systems 293-VE) at 200 ng per mL were added. The final concentration of FGF in the assay was 5 ng per mL and the final concentration of VEGF in the assays was 20 ng per mL. The growth factor free control wells had 20 μL per well of serum starvation media with the same amount of BSA as the wells with growth factors. The plates were returned to the incubator for an additional 22 hours.

BrdU ELISA

After 24 hour exposure to the test compounds, the cells were labeled with BrdU (Roche Biochemicals 1-647-229), by adding 20 μL per well of BrdU labeling reagent that has been diluted (1:100) in serum starvation medium. The plates were then returned to the incubator for 4 hours. The labeling medium was removed by draining the medium onto paper towels. The cells were fixed and DNA denatured by adding 200 μL of fixation/denaturation solution to each well and incubating at room temperature for 45 minutes. The fixation/denaturation solution was drained onto paper towels and to each well was added 100 μL of anti-BrdU-POD and the wells were incubated for 2 hours at room temperature. The antibody solution was removed and the wells were each washed 3-4 times with 300 μL PBS. 100 μL of the TMB substrate solution was added to each well and the wells were incubated at room temperature for 5-8 minutes. The reaction was then stopped by adding 100 μL per well of 1 M phosphoric acid. The plates were read at 450 nm with reference wavelength of 650 nm. The percent inhibition for each test compound was calculated by subtracting the absorbency of the blank (no cells) wells from all wells, then subtracting the division of the average absorbency of each test duplicate by the average of the controls from 1. The final product was then multiplied by 100 (% of inhibition=(1−average absorbency of test duplicate/average of control) 100). The IC₅₀ value is the concentration of test compound that inhibits by 50% BrdU labeling, and is a measure of inhibition of cell proliferation. The IC₅₀ is determined from the linear regression of a plot of the logarithm of the concentration versus percent inhibition.

The compounds of the present invention have VEGF-stimulated HUVEC assay IC₅₀ values less than 3 μM, preferably less than 1.5 μM, or FGF-stimulated HUVEC assay IC₅₀ values less than 5 μM, preferably less than 3.0 μM, even more preferably less than 2 μM. Most preferably, the compounds of the invention have VEGF-stimulated HUVEC assay IC₅₀ values less than 1.5 μM and FGF-stimulated HUVEC assay IC₅₀ values less than 2 μM.

### Example 28

Tablet Formulation

| Item | Ingredients | Mg/Tablet | | | | | |
|---|---|---|---|---|---|---|---|
| 1 | CompoundA * | 5 | 25 | 100 | 250 | 500 | 750 |
| 2 | Anhydrous Lactose | 103 | 83 | 35 | 19 | 38 | 57 |
| 3 | Croscarmellose | 6 | 6 | 8 | 16 | 32 | 48 |
| | Sodium | | | | | | |
| 4 | Povidone K30 | 5 | 5 | 6 | 12 | 24 | 36 |
| 5 | Magnesium Stearate | 1 | 1 | 1 | 3 | 6 | 9 |
| | Total Weight | 120 | 120 | 150 | 300 | 600 | 900 |

*Compound A represents a compound of the invention.

Manufacturing Procedure:

1. Mix Items 1, 2 and 3 in a suitable mixer for 15 minutes.

2. Granulate the powder mix from Step 1 with 20% Povidone K30 Solution (Item 4).

3. Dry the granulation from Step 2 at 50° C.

4. Pass the granulation from Step 3 through a suitable milling equipment.

5. Add the Item 5 to the milled granulation Step 4 and mix for 3 minutes.

6. Compress the granulation from Step 5 on a suitable press.

### Example 29

Capsule Formulation

| Item | Ingredients | mg/Capsule | | | | |
|---|---|---|---|---|---|---|
| 1 | Compound A* | 5 | 25 | 100 | 250 | 500 |
| 2 | Anhydrous Lactose | 159 | 123 | 148 | — | — |
| 3 | Corn Starch | 25 | 35 | 40 | 35 | 70 |
| 4 | Talc | 10 | 15 | 10 | 12 | 24 |
| 5 | Magnesium Stearate | 1 | 2 | 2 | 3 | 6 |
| | Total Fill Weight | 200 | 200 | 300 | 300 | 600 |

*Compound A represents a compound of the invention.

Manufacturing Procedure:

1. Mix Items 1, 2 and 3 in a suitable mixer for 15 minutes.

2. Add Items 4 & 5 and mix for 3 minutes.

3. Fill into a suitable capsule.

### Example 30

Injection Solution/Emulsion Preparation

| Item | Ingredient | mg/mL | |
|---|---|---|---|
| 1 | Compound A* | 1 | mg |
| 2 | PEG 400 | 10-50 | mg |
| 3 | Lecithin | 20-50 | mg |
| 4 | Soy Oil | 1-5 | mg |
| 5 | Glycerol | 8-12 | mg |
| 6 | Water q.s. | 1 | mL |

*Compound A represents a compound of the invention.

Manufacturing Procedure:

1. Dissolve item 1 in item 2.

2. Add items 3, 4 and 5 to item 6 and mix until dispersed, then homogenize.

3. Add the solution from step 1 to the mixture from step 2 and homogenize until the dispersion is translucent.

4. Sterile filter through a 0.2 μm filter and fill into vials.

### Example 31

Injection Solution/Emulsion Preparation

| Item | Ingredient | mg/mL | |
|---|---|---|---|
| 1 | Compound A* | 1 | mg |
| 2 | Glycofurol | 10-50 | mg |
| 3 | Lecithin | 20-50 | mg |
| 4 | Soy Oil | 1-5 | mg |
| 5 | Glycerol | 8-12 | mg |

| | | |
|---|---|---|
| 6 | Water | q.s. 1 mL |

*Compound A represents a compound of the invention.

Manufacturing Procedure:

1. Dissolve item 1 in item 2.

2. Add items 3, 4 and 5 to item 6 and mix until dispersed, then homogenize.

3. Add the solution from step 1 to the mixture from step 2 and homogenize until dispersion is translucent.

4. Sterile filter through a 0.2 μm filter and fill into vials.

While the invention has been illustrated by reference to specific and preferred embodiments, those skilled in the art will understand that variations and modifications may be made through routine experimentation and practice of the invention. Thus, the invention is intended not to be limited by the foregoing description, but to be defined by the appended claims and their equivalents.

1. A compound of formula: 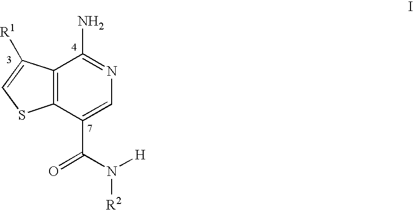
wherein
R¹ is selected from lower alkyl, and lower alkyl substituted with OR³, NR³R⁴, S(O)ₙR³, cycloalkyl, substituted cycloalkyl, heterocycle, substituted heterocycle, heteroaryl, or substituted heteroaryl;
R² is selected from H, lower alkyl, and lower alkyl substituted with OR⁵, OC(O)R⁵, NR⁵R⁶, S(O)ₙR⁵, aryl, substituted aryl, cycloalkyl, substituted cycloalkyl, heterocycle, substituted heterocycle, heteroaryl, or substituted heteroaryl;
R³ and R⁴ are independently selected from H, lower alkyl, lower alkyl substituted with aryl, aryl fused to a heterocycle or a substituted heterocycle, substituted aryl, heteroaryl, substituted heteroaryl, cycloalkyl, substituted cycloalkyl, heterocycle, or substituted heterocycle, aryl, aryl fused to a heterocycle or a substituted heterocycle, substituted aryl, heteroaryl′ heteroaryl fused to a heterocycle or a substituted heterocycle, substituted heteroaryl, heterocycle, heterocycle fused to an aryl, cycloalkyl and substituted cycloalkyl,
or, alternately, the group NR³R⁴ independently can form a ring having a total of 3 to 7 atoms, said ring atoms comprising in addition to the nitrogen to which R³ and R⁴ are bonded, carbon ring atoms, said carbon ring atoms optionally being replaced by one or more additional heteroatoms, and said ring atoms optionally being substituted by the group consisting of one or more lower alkyl, ═O, OR⁷, COR⁷, CO₂R⁷, CONR⁷R⁸, SOₙR⁷, and SO₂NR⁷R⁸;
R⁵ and R⁶ are independently selected from H, lower alkyl, and lower alkyl substituted with OR⁷, NR⁷R⁸, aryl, substituted aryl, heteroaryl, substituted heteroaryl, heterocycle, cycloalkyl, substituted cycloalkyl,
or, alternately, the group NR⁵R⁶ independently can form a ring having a total of 3 to 7 atoms, said ring atoms comprising in addition to the nitrogen to which R⁵ and R⁶ are bonded, carbon ring atoms, said carbon ring atoms optionally being replaced by one or more additional heteroatoms, and said ring atoms optionally being substituted by the group consisting of one or more lower alkyl, ═O, OR⁷, NR⁷R⁸, COR⁷, CO₂R⁷, CONR⁷R⁸, SOₙR⁷, and SO₂NR⁷R⁸;
R⁷ and R⁸ are independently selected from H, and lower alkyl, or, alternatively, the group NR⁷R⁸ independently can form a ring having a total of 3 to 7 atoms, said ring atoms comprising in addition to the nitrogen to which R⁷ and R⁸ are bonded, carbon ring atoms, said carbon ring atoms optionally being replaced by one or more additional heteroatoms, and said ring atoms optionally being substituted by the group consisting of one or more lower alkyl, ═O, or OR⁹;
R⁹ is H or lower alkyl; and
n is 0, 1 or 2;
wherein,
substituted aryl and substituted heteroaryl are aryl and heteroaryl that are substituted with one or more groups independently selected from lower alkyl, OR⁷, NR⁷R⁸, COR⁷, CO₂R⁷₇ CONR⁷R⁸, SO₂NR⁷R⁸₇ SOₙR⁷₇ CN, NO₂, and halogen; and
substituted cycloalkyl and substituted heterocycle are cycloalkyl and heterocycle that are substituted with one or more groups independently selected from lower alkyl, ═O, OR⁷, NR⁷R⁸, COR⁷, CO₂R⁷, CONR⁷R⁸, SO₂NR⁷R⁸, SOₙR⁷, and CN;
or a pharmaceutically acceptable salt or ester thereof.

2. The compound of claim 1 wherein R¹ is lower alkyl substituted with OR³.

3. The compound of claim 2 wherein R³ is selected from aryl, aryl substituted with halogen, and aryl fused to a heterocycle.

4. The compound of claim 3 wherein the halogen is Br, Cl or F.

5. The compound of claim 2 wherein R³ groups is selected from heteroaryl and heteroaryl substituted with OR⁷.

6. The compound of claim 1 wherein R¹ is lower alkyl substituted with NR³R⁴.

7. The compound of claim 6 wherein the group NR³R⁴ forms a ring having a total of 3 to 7 ring atoms comprising in addition to the nitrogen to which R³ and R⁴ are bonded, carbon ring atoms, said carbon ring atoms optionally being replaced by one or more additional heteroatoms, and said ring atoms optionally being substituted by the group consisting of one or more lower alkyl, ═O, OR⁷, COR⁷, CO₂R⁷, CONR⁷R⁸, SOₙR⁷, and SO₂NR⁷R⁸, preferably OR⁷.

8. The compound of claim 1 wherein R¹ is lower alkyl substituted with S(O)ₙR³, and R³ is lower alkyl, heterocycle, heterocycle fused to an aryl, aryl, substituted aryl, and aryl fused to a heterocycle.

9. The compound of claim 1 wherein R¹ is lower alkyl substituted with cycloalkyl.

10. The compound of claim 1 wherein R¹ is lower alkyl substituted with substituted cycloalkyl.

11. The compound of claim 1 wherein R¹ is lower alkyl substituted with heterocycle.

12. The compound of claim 1 wherein R¹ is lower alkyl substituted with substituted heterocycle.

13. The compound of claim 1 wherein R¹ is lower alkyl substituted with heteroaryl.

14. The compound of claim 1 wherein R¹ is lower alkyl substituted with substituted heteroaryl.

15. The compound of claim 1 wherein R¹ is lower alkyl.

16. The compound of claim 1 wherein R² is lower alkyl substituted with OR⁵ and R⁵ is lower alkyl substituted with NR⁷R⁸.

17. The compound of claim 1 wherein R² is lower alkyl substituted with NR⁵R⁶.

18. The compound of claim 1 wherein R² is lower alkyl substituted with OR⁵ where R⁵ is hydrogen.

19. The compound of claim 1 wherein R² is lower alkyl substituted with OR⁵ where R⁵ is lower alkyl substituted by OR⁷.

20. The compound of claim 17 wherein the group NR⁵R⁶ forms a ring having a total of 3 to 7 ring atoms, said ring atoms comprising in addition to the nitrogen to which R⁵ and R⁶ are bonded, carbon ring atoms, said carbon ring atoms optionally being replaced by one or more additional heteroatoms, and said ring atoms optionally being substituted by the group consisting of one or more lower alkyl, ═O, OR⁷, NR⁷R⁸, COR⁷, CO₂R⁷, CONR⁷R⁸, SOₙR⁷, and SO₂NR⁷R⁸.

21. The compound of claim 1 wherein R² is lower alkyl substituted with SO₍ₙ₎R⁵.

22. The compound of claim 1 wherein R² is lower alkyl.

23. The compound of claim 1 wherein R² is lower alkyl substituted with one OH group or one NR⁵R⁶ group.

24. The compound of claim 1 wherein R² is H.

25. A compound of formula I 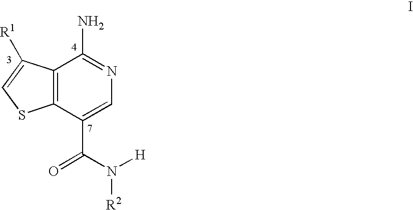
wherein
R¹ is lower alkyl substituted with OR³;
R² is H or lower alkyl substituted with one OR⁵ group or one NR⁵R⁶ group;
R³ is aryl substituted with halogen or OR⁷, or is aryl fused to a heterocycle;
R⁵ and R⁶ are independently H or lower alkyl, or alternatively, the group NR⁵R⁶ independently can form a ring having a total of from 3 to 6 atoms, said ring atoms comprising in addition to the nitrogen to which R⁵ and R⁶ are bonded, carbon ring atoms, said carbon ring atoms optionally being replaced by one additional heteroatoms selected from N or O, and said ring atoms optionally being substituted by OR⁷; and
R⁷ is H or lower alkyl;
or a pharmaceutically acceptable salt or ester thereof.

26. The compound of claim 1 selected from the group consisting of:
4-Amino-3-(4-bromo-2,6-difluoro-phenoxymethyl)-thieno[3,2-c]pyridine-7-carboxylic acid (2-hydroxy-ethyl)-amide,
4-Amino-3-(2-chloro-4-methoxy-phenoxymethyl)-thieno[3,2-c]pyridine-7-carboxylic acid (2-hydroxy-ethyl)-amide,
4-Amino-3-(benzo[1,3]dioxol-5-yloxymethyl)-thieno[3,2-c]pyridine-7-carboxylic acid (2-hydroxy-ethyl)-amide,
4-Amino-3-(4-bromo-phenoxymethyl)-thieno[3,2-c]pyridine-7-carboxylic acid (2-hydroxy-ethyl)-amide, and
4-Amino-3-phenoxymethyl-thieno[3,2-c]pyridine-7-carboxylic acid (2-hydroxy-ethyl)-amide.

27. The compound of claim 1 selected from the group consisting of:
rac-4-Amino-3-(4-bromo-phenoxymethyl)-thieno[3,2-c]pyridine-7-carboxylic acid (2-hydroxy-1-methyl-ethyl)-amide,
rac-4-Amino-3-(4-bromo-phenoxymethyl)-thieno[3,2-c]pyridine-7-carboxylic acid (2-hydroxy-propyl)-amide, and
rac-4-Amino-3-(4-bromo-phenoxymethyl)-thieno[3,2-c]pyridine-7-carboxylic acid (2,3-dihydroxy-propyl)-amide.

28. The compound of claim 1 selected from the group consisting of:
4-Amino-3-(4-bromo-phenoxymethyl)-thieno[3,2-c]pyridine-7-carboxylic acid (2-hydroxy-1,1-dimethyl-ethyl)-amide,
4-Amino-3-(4-bromo-phenoxymethyl)-thieno[3,2-c]pyridine-7-carboxylic acid (2-hydroxy-1-hydroxymethyl-ethyl)-amide,
4-Amino-3-(4-bromo-phenoxymethyl)-thieno[3,2-c]pyridine-7-carboxylic acid (2-diethylamino-ethyl)-amide,
4-Amino-3-(4-bromo-phenoxymethyl)-thieno[3,2-c]pyridine-7-carboxylic acid (4-pyrrolidin-1-yl-butyl)-amide,
4-Amino-3-(4-bromo-phenoxymethyl)-thieno[3,2-c]pyridine-7-carboxylic acid (4-pyrrolidin-1-yl-butyl)-amide hydrochloride salt, and
4-Amino-3-(4-bromo-phenoxymethyl)-thieno[3,2-c]pyridine-7-carboxylic acid (4-pyrrolidin-1-yl-butyl)-amide methanesulfonic acid salt.

29. The compound of claim 1 selected from the group consisting of:
4-Amino-3-(4-bromo-phenoxymethyl)-thieno[3,2-c]pyridine-7-carboxylic acid (2-morpholin-4-yl-ethyl)-amide,
4-Amino-3-(4-bromo-phenoxymethyl)-thieno[3,2-c]pyridine-7-carboxylic acid (2-morpholin-4-yl-ethyl)-amide hydrochloride salt,
4-Amino-3-(4-bromo-phenoxymethyl)-thieno[3,2-c]pyridine-7-carboxylic acid (3-dimethylamino-2,2-dimethyl-propyl)-amide,
rac-4-Amino-3-(4-bromo-phenoxymethyl)-thieno[3,2-c]pyridine-7-carboxylic acid (5-diethylamino-1-methyl-pentyl)-amide,
4-Amino-3-(4-bromo-phenoxymethyl)-thieno[3,2-c]pyridine-7-carboxylic acid amide, and
4-Amino-3-(4-bromo-phenoxymethyl)-thieno[3,2-c]pyridine-7-carboxylic acid [2-(2-pyrrolidin-1-yl-ethoxy)-ethyl]-amide.

30. The compound of claim 1 selected from the group consisting of:
4-Amino-3-(4-bromo-phenoxymethyl)-thieno[3,2-c]pyridine-7-carboxylic acid [4-(3-methoxy-pyrrolidin-1-yl)-butyl]-amide,
4-Amino-3-(4-bromo-phenoxymethyl)-thieno[3,2-c]pyridine-7-carboxylic acid (4-piperidin-1-yl-butyl)-amide,
4-Amino-3-(4-bromo-phenoxymethyl)-thieno[3,2-c]pyridine-7-carboxylic acid [2-(2-piperidin-1-yl-ethoxy)-ethyl]-amide,
4-Amino-3-(4-bromo-phenoxymethyl)-thieno[3,2-c]pyridine-7-carboxylic acid [4-(3-methoxy-piperidin-1-yl)-butyl]-amide,
4-Amino-3-(4-bromo-phenoxymethyl)-thieno[3,2-c]pyridine-7-carboxylic acid (4-morpholin-4-yl-butyl)-amide,
4-Amino-3-(4-bromo-phenoxymethyl)-thieno[3,2-c]pyridine-7-carboxylic acid [2-(2-morpholin-4-yl-ethoxy)-ethyl]-amide, and
4-Amino-3-(4-bromo-phenoxymethyl)-thieno[3,2-c]pyridine-7-carboxylic acid [4-(4-methoxy-piperidin-1-yl)-butyl]-amide,
4-Amino-3-(4-bromo-phenoxymethyl)-thieno[3,2-c]pyridine-7-carboxylic acid [3-(2,3-dihydroxy-propoxy)-propyl]-amide.

31. A pharmaceutical composition comprising a therapeutically effective amount of a compound of formula I 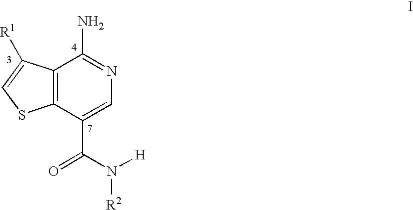
wherein
R¹ is selected from lower alkyl, and lower alkyl substituted with OR³, NR³R⁴, S(O)ₙR³, cycloalkyl, substituted cycloalkyl, heterocycle, substituted heterocycle, heteroaryl, or substituted heteroaryl;
R² is selected from H, lower alkyl, and lower alkyl substituted with OR⁵, OC(O)R⁵, NR⁵R⁶, S(O)ₙR⁵, aryl, substituted aryl, cycloalkyl, substituted cycloalkyl, heterocycle, substituted heterocycle, heteroaryl, or substituted heteroaryl;
R³ and R⁴ are independently selected from
H,
lower alkyl,
lower alkyl substituted with aryl, aryl fused to a heterocycle or a substituted heterocycle, substituted aryl, heteroaryl, substituted heteroaryl, cycloalkyl, substituted cycloalkyl, heterocycle, or substituted heterocycle,
aryl,
aryl fused to a heterocycle or a substituted heterocycle,
substituted aryl,
heteroaryl,
heteroaryl fused to a heterocycle or a substituted heterocycle
substituted heteroaryl,
cycloalkyl, and
substituted cycloalkyl,
or, alternately, the group NR³R⁴ independently can form a ring having a total of 3 to 7 atoms, said ring atoms comprising in addition to the nitrogen to which R³ and R⁴ are bonded, carbon ring atoms, said carbon ring atoms optionally being replaced by one or more additional heteroatoms, and said ring atoms optionally being substituted by the group consisting of one or more lower alkyl, ═O, OR⁷, COR⁷, CO₂R⁷, CONR⁷R⁸, SOₙR⁷, and SO₂NR⁷R⁸;
R⁵ and R⁶ are independently selected from
H,
lower alkyl, and
lower alkyl substituted with OR⁷, NR⁷R⁸, aryl, substituted aryl, heteroaryl, substituted heteroaryl, heterocycle, cycloalkyl, substituted cycloalkyl,
or, alternately, the group NR⁵R⁶ independently can form a ring having a total of 3 to 7 atoms, said ring atoms comprising in addition to the nitrogen to which R⁵ and R⁶ are bonded, carbon ring atoms, said carbon ring atoms optionally being replaced by one or more additional heteroatoms, and said ring atoms optionally being substituted by the group consisting of one or more lower alkyl, ═O, OR⁷, NR⁷R⁸, COR⁷, CO₂R⁷, CONR⁷R⁸, SOₙR⁷, and SO₂NR⁷R⁸;
R⁷ and R⁸ are independently selected from
H, and
lower alkyl,
or, alternatively, the group NR⁷R⁸ independently can form a ring having a total of 3 to 7 atoms, said ring atoms comprising in addition to the nitrogen to which R⁷ and R⁸ are bonded, carbon ring atoms, said carbon ring atoms optionally being replaced by one or more additional heteroatoms, and said ring atoms optionally being substituted by the group consisting of one or more lower alkyl, ═O, or OR⁹;
R⁹ is H or lower alkyl; and
n is 0, 1 or 2;
wherein,
substituted aryl and substituted heteroaryl are aryl and heteroaryl that are substituted with one or more groups independently selected from lower alkyl, OR⁷, NR⁷R⁸, COR⁷, CO₂R⁷, CONR⁷R⁸, SO₂NR⁷R⁸, SOₙR⁷, CN, NO₂, and halogen; and
substituted cycloalkyl and substituted heterocycle are cycloalkyl and heterocycle that are substituted with one or more groups independently selected from lower alkyl, ═O, OR⁷, NR⁷R⁸, COR⁷, CO₂R⁷, CONR⁷R⁸, SO₂NR⁷R⁸, SOₙR⁷, and CN;
or a pharmaceutically acceptable salt or ester thereof;
and a pharmaceutically acceptable carrier or excipient.

32. A method for treating cancer comprising administering to a patient in need of such treatment a therapeutically effective amount of a compound of formula I 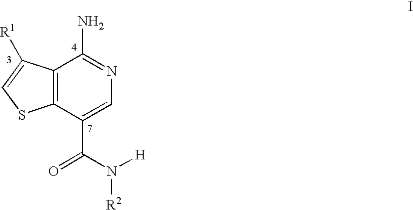
wherein
R¹ is selected from
lower alkyl, and
lower alkyl substituted with OR³, NR³R⁴, S(O)NR³ cycloalkyl, substituted cycloalkyl, heterocycle, substituted heterocycle, heteroaryl, or substituted heteroaryl;
R² is selected from
H,
lower alkyl, and
lower alkyl substituted with OR⁵, OC(O)R⁵, NR⁵R⁶, S(O)NR⁵, aryl, substituted aryl, cycloalkyl, substituted cycloalkyl, heterocycle, substituted heterocycle, heteroaryl, or substituted heteroaryl;
R³ and R⁴ are independently selected from
H,
lower alkyl,
lower alkyl substituted with aryl, aryl fused to a heterocycle or a substituted heterocycle, substituted aryl, heteroaryl, substituted heteroaryl, cycloalkyl, substituted cycloalkyl, heterocycle, or substituted heterocycle,
aryl,
aryl fused to a heterocycle or a substituted heterocycle,
substituted aryl,
heteroaryl,
heteroaryl fused to a heterocycle or a substituted heterocycle
substituted heteroaryl,
cycloalkyl, and
substituted cycloalkyl,
or, alternately, the group NR³R⁴ independently can form a ring having a total of 3 to 7 atoms, said ring atoms comprising in addition to the nitrogen to which R³ and R⁴ are bonded, carbon ring atoms, said carbon ring atoms optionally being replaced by one or more additional heteroatoms, and said ring atoms optionally being substituted by the group consisting of one or more lower alkyl, ═O, OR⁷, COR⁷, CO₂R⁷, CONR⁷R⁸, SOₙR⁷, and SO₂NR⁷R⁸;
R⁵ and R⁶ are independently selected from
H,
lower alkyl, and
lower alkyl substituted with OR⁷, NR⁷R⁸, aryl, substituted aryl, heteroaryl, substituted heteroaryl, heterocycle, cycloalkyl, substituted cycloalkyl,
or, alternately, the group NR⁵R⁶ independently can form a ring having a total of 3 to 7 atoms, said ring atoms comprising in addition to the nitrogen to which R⁵ and R⁶ are bonded, carbon ring atoms, said carbon ring atoms optionally being replaced by one or more additional heteroatoms, and said ring atoms optionally being substituted by the group consisting of one or more lower alkyl, ═O, OR⁷, NR⁷R⁸, COR⁷, CO₂R⁷, CONR⁷R⁸, SOₙR⁷, and SO₂NR⁷R⁸;
R⁷ and R⁸ are independently selected from
H, and
lower alkyl,
or, alternatively, the group NR⁷R⁸ independently can form a ring having a total of 3 to 7 atoms, said ring atoms comprising in addition to the nitrogen to which R⁷ and R⁸ are bonded, carbon ring atoms, said carbon ring atoms optionally being replaced by one or more additional heteroatoms, and said ring atoms optionally being substituted by the group consisting of one or more lower alkyl, ═O, or OR⁹;
R⁹ is H or lower alkyl; and
n is 0, 1 or 2;
wherein,
substituted aryl and substituted heteroaryl are aryl and heteroaryl that are substituted with one or more groups independently selected from lower alkyl, OR⁷, NR⁷R⁸, COR⁷, CO₂R⁷, CONR⁷R⁸, SO₂NR⁷R⁸, SOₙR⁷, CN, NO₂, and halogen; and
substituted cycloalkyl and substituted heterocycle are cycloalkyl and heterocycle that are substituted with one or more groups independently selected from lower alkyl, ═O, OR⁷, NR⁷R⁸, COR⁷, CO₂R⁷, CONR⁷R⁸, SO₂NR⁷R⁸, SOₙR⁷, and CN;
or a pharmaceutically acceptable salt or ester thereof.

33. A method of controlling cancer comprising administering to a patient in need of such treatment a therapeutically effective amount of a compound of formula I 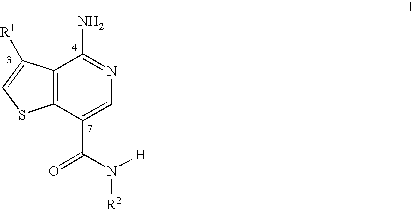
wherein
R¹ is selected from
lower alkyl, and
lower alkyl substituted with OR³, NR³R⁴, S(O)NR³, cycloalkyl, substituted cycloalkyl, heterocycle, substituted heterocycle, heteroaryl, or substituted heteroaryl;
R² is selected from
H,
lower alkyl, and
lower alkyl substituted with OR⁵, OC(O)R⁵, NR⁵R⁶, S(O)ₙR⁵, aryl, substituted aryl, cycloalkyl, substituted cycloalkyl, heterocycle, substituted heterocycle, heteroaryl, or substituted heteroaryl;
R³ and R⁴ are independently selected from
H,
lower alkyl,
lower alkyl substituted with aryl, aryl fused to a heterocycle or a substituted heterocycle, substituted aryl, heteroaryl, substituted heteroaryl, cycloalkyl, substituted cycloalkyl, heterocycle, or substituted heterocycle,
aryl,
aryl fused to a heterocycle or a substituted heterocycle,
substituted aryl,
heteroaryl,
heteroaryl fused to a heterocycle or a substituted heterocycle
substituted heteroaryl,
cycloalkyl, and
substituted cycloalkyl,
or, alternately, the group NR³R⁴ independently can form a ring having a total of 3 to 7 atoms, said ring atoms comprising in addition to the nitrogen to which R³ and R⁴ are bonded, carbon ring atoms, said carbon ring atoms optionally being replaced by one or more additional heteroatoms, and said ring atoms optionally being substituted by the group consisting of one or more lower alkyl, ═O, OR⁷, COR⁷, CO₂R⁷, CONR⁷R⁸, SOₙR⁷, and SO₂NR⁷R⁸;
R⁵ and R⁶ are independently selected from
H,
lower alkyl, and
lower alkyl substituted with OR⁷, NR⁷R⁸, aryl, substituted aryl, heteroaryl, substituted heteroaryl, heterocycle, cycloalkyl, substituted cycloalkyl,
or, alternately, the group NR⁵R⁶ independently can form a ring having a total of 3 to 7 atoms, said ring atoms comprising in addition to the nitrogen to which R⁵ and R⁶ are bonded, carbon ring atoms, said carbon ring atoms optionally being replaced by one or more additional heteroatoms, and said ring atoms optionally being substituted by the group consisting of one or more lower alkyl, ═O, OR⁷, NR⁷R⁸, COR⁷, CO₂R⁷, CONR⁷R⁸, SOₙR⁷, and SO₂NR⁷R⁸;
R⁷ and R⁸ are independently selected from
H, and
lower alkyl,
or, alternatively, the group NR⁷R⁸ independently can form a ring having a total of 3 to 7 atoms, said ring atoms comprising in addition to the nitrogen to which R⁷ and R⁸ are bonded, carbon ring atoms, said carbon ring atoms optionally being replaced by one or more additional heteroatoms, and said ring atoms optionally being substituted by the group consisting of one or more lower alkyl, ═O, or OR⁹;
R⁹ is H or lower alkyl; and
n is 0, 1 or 2;
wherein,
substituted aryl and substituted heteroaryl are aryl and heteroaryl that are substituted with one or more groups independently selected from lower alkyl, OR⁷, NR⁷R⁸, COR⁷, CO₂R⁷, CONR⁷R⁸, SO₂NR⁷R⁸, SOₙR⁷, CN, NO₂, and halogen; and
substituted cycloalkyl and substituted heterocycle are cycloalkyl and heterocycle that are substituted with one or more groups independently selected from lower alkyl, ═O, OR⁷, NR⁷R⁸, COR⁷, CO₂R⁷, CONR⁷R⁸, SO₂NR⁷R⁸, SOₙR⁷, and CN;
or a pharmaceutically acceptable salt or ester thereof.

34. The method of claim 32 wherein the cancer is breast, lung, colon or prostate cancer.

35. The method of claim 33 wherein the cancer is breast, lung, colon cancer or prostate.

36. A compound selected from the group:
3-Methyl-5H-thieno[3,2-c]pyridin-4-one,
7-Iodo-3-methyl-5H-thieno[3,2-c]pyridin-4-one,
3-Methyl-4-oxo-4,5-dihydro-thieno[3,2-c]pyridine-7-carboxylic acid ethyl ester,
4-Chloro-3-methyl-thieno[3,2-c]pyridine-7-carboxylic acid ethyl ester,
3-Bromomethyl-4-chloro-thieno[3,2-c]pyridine-7-carboxylic acid ethyl ester,
3-(4-Bromo-2,6-difluoro-phenoxymethyl)-4-chloro-thieno[3,2-c]pyridine-7-carboxylic acid ethyl ester, and
4-Amino-3-(4-bromo-2,6-difluoro-phenoxymethyl)-thieno[3,2-c]pyridine-7-carboxylic acid ethyl ester.

37. A compound selected from the group:
4-Amino-3-(2-chloro-4-methoxy-phenoxymethyl)-thieno[3,2-c]pyridine-7-carboxylic acid ethyl ester,
3-(4-Bromo-phenoxymethyl)-4-chloro-thieno[3,2-c]pyridine-7-carboxylic acid ethyl ester,
4-Amino-3-(4-bromo-phenoxymethyl)-thieno[3,2-c]pyridine-7-carboxylic acid ethyl ester,
4-Chloro-3-phenoxymethyl-thieno[3,2-c]pyridine-7-carboxylic acid ethyl ester,
4-Amino-3-phenoxymethyl-thieno[3,2-c]pyridine-7-carboxylic acid ethyl ester, and
4-Amino-3-(4-bromo-phenoxymethyl)-thieno[3,2-c]pyridine-7-carboxylic acid.
